(19) United States
(12) Patent Application Publication
(10) Pub. No.: US 2024/0376042 A1
(43) Pub. Date: Nov. 14, 2024

(54) ORGANIC COMPOUNDS AND APPLICATIONS THEREOF IN ORGANIC ELECTRONIC DEVICES

(71) Applicant: Zhejiang Brilliant Optoelectronic Technology Co.,Ltd., Taizhou (CN)

(72) Inventors: Jiahui TAN, Taizhou (CN); Xiang CHEN, Taizhou (CN); Junyou PAN, Taizhou (CN)

(73) Assignee: Zhejiang Brilliant Optoelectronic Technology Co.,Ltd., Taizhou (CN)

(21) Appl. No.: 18/405,257

(22) Filed: Jan. 5, 2024

Related U.S. Application Data
(63) Continuation of application No. PCT/CN2022/104266, filed on Jul. 7, 2022.

(30) Foreign Application Priority Data
Jul. 7, 2021 (CN) 202110767608.7

(51) Int. Cl. C07C 255/51; C07C 255/61; C07D 401/08; C07D 403/08; C07D 407/08; C07D 409/08; C07D 413/08; C07D 417/08; C07D 417/14; C07D 487/04; C07D 519/00; H10K 50/15; H10K 85/60

(52) U.S. Cl. CPC C07C 255/51; C07C 255/61; C07D 401/08; C07D 403/08; C07D 407/08; C07D 409/08; C07D 413/08; C07D 417/08; C07D 417/14; C07D 487/04; C07D 519/00; H10K 50/15; H10K 85/654; H10K 85/6572

(57) ABSTRACT

Disclosed are organic compounds including a structure of formula (I-1). Also provided are mixtures containing the organic compounds, and at least one organic functional material. Further provided are organic electronic devices containing a functional layer, the functional layer comprises the organic compounds. The present disclosure can achieve better device performance through device structure optimization, especially to achieve high-performance OLED devices, which provides better material and preparation technology options for full-color display and lighting applications.

### CROSS-REFERENCE TO RELATED APPLICATIONS

The present application is a continuation of International Application No. PCT/CN2022/104266, filed on Jul. 7, 2022, which claims priority to Chinese Patent Application No. 202110767608.7, filed on Jul. 7, 2021. All of the aforementioned applications are incorporated herein by reference in their entireties.

### TECHNICAL FIELD

The present disclosure relates to the field of electroluminescent material, and in particularly to an organic compound, a mixture, and the applications thereof in organic electronic devices, particularly in organic electroluminescent devices.

### BACKGROUND

Due to the diversity of synthesis, realizability in low manufacturing cost, and excellent optical and electrical properties, organic light-emitting diodes (OLEDs) have great potential for the realization of optoelectronic devices, such as in flat-panel displays and lighting applications.

The organic electroluminescent phenomenon refers to a phenomenon of converting electrical energy to photonic energy with organic substance. An organic electroluminescent element utilizing the organic electroluminescent phenomenon usually has a structure comprising an anode, a cathode, and an organic layer therebetween. In order to improve the efficiency and lifetime of the organic electroluminescent element, the organic layer has a multi-layer structure, and each layer comprises different organic substances. For example, each layer can be a hole-injection layer, a hole-transport layer, a light-emitting layer, an electron-transport layer, an electron-injection layer, etc. When a voltage is applied between two electrodes of such an organic electroluminescent element, holes are injected into the organic layer from the anode, electrons are injected into an organic layer from the cathode; and an exciton is formed when an injected hole and an injected electron recombine in the emission layer. The exciton emits light when it transitions back to the ground state. The organic electroluminescent element has characteristics of self-emission, high luminance, high efficiency, low driving voltage, wide viewing angle, high contrast, high responsivity, etc.

Aromatic amine derivatives are known to be applied as hole-transport materials in organic light-emitting devices. In organic light-emitting devices using aromatic amine derivatives as hole-transport materials, the applied voltage is increased to obtain sufficient luminescent brightness, which results in a short device lifetime and high power consumption. In order to solve these problems, Karl Leo et al. proposed the concept of p-type doping (U.S. Pat. No. 7,074,500B2), in which electron-accepting compounds are doped as p-doped materials in the hole-transport material to increase the carrier concentration and thus reduce the operating voltage. Methods of fabricating a doped hole-injection layer or an isolation layer with an electron-accepting compound (e.g., lewis acid) have been proposed in the patent documents 1-5 (patent document 1: JP4023204B2; patent document 2: JP3978976B2; patent document 3: CN1475035A; patent document 4: US20050255334A1; patent document 5: U.S. Pat. No. 7,697,056B2). However, the electron-accepting compounds used therein are unstable during the manufacturing or driving (e.g. heat resistance) of the organic light-emitting devices, leading to the short device lifetime. In addition, a representative electron-accepting compound, F₄TCNQ (2,3,5,6-Tetrafluoro-7,7,8,8-tetracyanoquinodimethane) described in patent documents 3 and 4, has a small molecular weight and fluorine substituents, resulting in too good sublimation property so that to diffuse into the equipments during the vacuum deposition, therefore contaminating the equipments or devices.(patent document 5). The problem of the another typical compound, HAT-CN, is that the deposition thickness is limited by current leakage and crystallization.

Furthermore, the electrodes are usually made from metals or metal oxides, and the resulting interface between the electrodes (inorganic materials) and the charge-injection layer (organic materials) is unstable. Based on the unstable interface, the device performance may be significantly decreased by externally applied heat, internally generated heat, or an electric field applied to the device. Therefore, it is necessary to develop new materials capable of forming stable interfaces with electrodes and having high charge transport capability.

### SUMMARY

The In one aspect, the present disclosure provides an organic compound comprising a structure of formula (I-1):

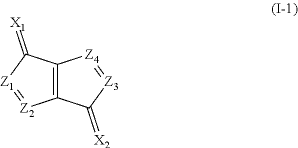

Where Z₁ to Z₄ are independently selected from CR₁ or N; X₁ and X₂ are independently selected from CR₂R₃, NR₂, O, S, S(═O)₂, SiR₂R₃, a substituted/unsubstituted aromatic group containing 6 to 60 carbon atoms, a substituted/unsubstituted heteroaromatic group containing 5 to 60 ring atoms, or a non-aromatic ring system containing 3 to 30 ring atoms.

R₁ in multiple occurrences, may be independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof.

each of R₂ and R₃ at each occurrence is independently selected from the group consisting of a nitro group, a nitroso group, —CF₃, —Cl, —Br, —F, —I, a cyano group, an alkoxy group, a C₁-C₂₀ ketone group, a C₂-C₂₀ alkoxycarbonyl group, a C₇-C₂₀ aryloxycarbonyl group, a carbamoyl group, a haloformyl group, a formyl group, an isocyano group, an isocyanate group, a thiocyanate group, an isothiocyanate group, a hydroxyl group, a substituted/unsubstituted aromatic or heteroaromatic group containing 5 to 60 ring atoms, an aryloxy or heteroaryloxy group containing 5 to 60 ring atoms, and any combination thereof.

In the organic compound as described herein, at least one of X₁, X₂, Z₁, Z₂, Z₃, or Z₄ comprises a cyano group.

In the organic compound as described herein, at least one of X₁ or X₂ comprises a cyano group.

In another aspect, the present disclosure also provides a mixture comprising an organic compound as described herein, and at least one organic functional material, the at least one organic functional material is selected from a hole-injection material, a hole-transport material, an electron-transport material, an electron-injection material, an electron-blocking material, a hole-blocking material, an emitting material, a host material, or an organic dye.

In yet another aspect, the present disclosure further provides a formulation comprising an organic compound or a mixture as described herein, and at least one organic solvent.

In yet another aspect, the present disclosure further provides an organic electronic device comprising a functional layer, the functional layer comprises an organic compound or a mixture as described herein, or is prepared using a formulation as described herein.

Beneficial effect: when the organic compound as described herein is used as a material for an organic EL element, especially for a hole-injection layer, low driving voltage and long lifetime of the device can be realized. In addition, the organic compound as described herein has a high molecular weight and does not contaminate the equipments or devices during the sublimation.

### DETAILED DESCRIPTION OF THE EMBODIMENTS

The present disclosure is to provide an organic compound, a mixture, an organic electronic device, and the applications thereof, so that their applications in electronic devices can reduce the driving voltage and prolong the device lifetime.

In order to make the objects, technical solutions, and effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink”, and “ink” have the same meaning, and they are interchangeable with each other.

As used herein, the terms “host material”, “matrix material” have the same meaning, and they are interchangeable with each other.

As used herein, the term “substituted” means that a hydrogen atom of the compound is substituted.

As used herein, “the number of ring atoms” means that the number of atoms constituting the ring itself of a structural compound (e. g., a monocyclic compound, a fused ring compound, a cross-linked compound, a carbocyclic compound, and a heterocyclic compound) by covalent bonding. When the ring is substituted with a substituent, the atoms contained in the substituent are not included in the ring atoms. The above rule applies for all cases without further specific description. For example, the number of ring atoms of a benzene ring is 6, the number of ring atoms of a naphthalene ring is 10, and the number of ring atoms of a thienyl group is 5.

The term “aromatic group” refers to a hydrocarbon group containing aromatic ring. The term “heteroaromatic group” refers to a heteroaromatic group consisting of at least one heteroatom. The heteroatom is preferably selected from Si, N, P, O, S and/or Ge, particularly preferably selected from Si, N, P, O and/or S. The term “fused-ring aromatic group” refers to an aromatic group containing two or more rings, in which two carbon atoms are shared by two adjacent rings, i. e., fused rings. The term “fused heterocyclic aromatic group” refers a fused aromatic hydrocarbon group containing at least one heteroatom. For the purposes of the present disclosure, the aromatic groups or heteroaromatic groups comprise not only aromatic ring systems, but also non-aromatic ring systems. Therefore, systems such as pyridine, thiophene, pyrrole, pyrazole, triazole, imidazole, oxazole, oxadiazole, thiazole, tetrazole, pyrazine, pyridazine, pyrimidine, triazine, carbene, and the like is also considered be aromatic groups or heterocyclic aromatic groups for the purposes of this disclosure. For the purposes of the present disclosure, the fused-ring aromatic or fused heterocyclic aromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be fused-ring aromatic ring systems for the purposes of this invention.

In embodiments of the present disclosure, the energy level structure of the organic material, singlet energy level (S1), triplet energy level (T1), highest occupied molecular orbital (HOMO), and lowest unoccupied molecular orbital (LUMO) play key roles. The determination of these energy levels is introduced as follows.

HOMO and LUMO energy levels can be measured by optoelectronic effect, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT) are becoming effective method for calculating the molecular orbital energy levels.

The singlet energy level (S1) of the organic material can be determined by the emission spectrum, the triplet energy level (T1) of an organic material can be measured by low-temperature time-resolved spectroscopy, S1 and T1 can also be calculated by quantum simulation (for example, by Time-dependent DFT), for instance with the commercial software Gaussian 09W (Gaussian Inc.), the specific simulation method can be found in WO2011141110 or as described below in the following embodiments. ΔE_{ST} is defined as (S1-T1).

It should be noted that the absolute values of HOMO, LUMO, Si and T1 may vary depending on the measurement method or calculation method used. Even for the same method, different ways of evaluation, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, Si and T1 are based on the Time-dependent DFT simulation, which however should not exclude the applications of other measurement or calculation methods.

In the invention, (HOMO−1) is defined as the energy level of the second highest occupied molecular orbital, (HOMO−2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on. ΔLUMO is defined as (LUMO+1)-LUMO, and ΔHOMO is defined as HOMO-(HOMO−1).

In one aspect, the present disclosure provides an organic compound comprising a structure of formula (I-1):

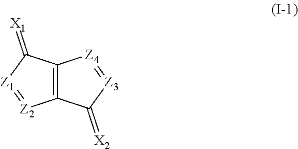

Where Z₁-Z₄ are independently selected from CR₁ or N; X₁ and X₂ are independently selected from CR₂R₃, NR₂, O, S, S(═O)₂, SiR₂R₃, a substituted/unsubstituted aromatic group containing 6 to 60 carbon atoms, a substituted/unsubstituted heteroaromatic group containing 5 to 60 ring atoms, or a non-aromatic ring system containing 3 to 30 ring atoms, and at least one of X₁, X₂, Z₁, Z₂, Z₃, or Z₄ comprises a cyano group; R₁ in multiple occurrences, may be independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; each of R₂ and R₃ at each occurrence is independently selected from the group consisting of a nitro group, a nitroso group, —CF₃, —Cl, —Br, —F, —I, a cyano group, an alkoxy group, a C₁-C₂₀ ketone group, a C₂-C₂₀ alkoxycarbonyl group, a C₇-C₂₀ aryloxycarbonyl group, a carbamoyl group, a haloformyl group, a formyl group, an isocyano group, an isocyanate group, a thiocyanate group, an isothiocyanate group, a hydroxyl group, a substituted/unsubstituted aromatic or heteroaromatic group containing 5 to 60 ring atoms, an aryloxy or heteroaryloxy group containing 5 to 60 ring atoms, and any combination thereof.

In some embodiments, R₁ at each occurrence is independently selected from the group consisting of -D, a C₁-C₂₀ linear alkyl group, a C₃-C₂₀ branched/cyclic alkyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a substituted/unsubstituted aromatic or heteroaromatic group containing 5 to 60 ring atoms, and any combination thereof; the adjacent R is may be bonded with each other to form a substituted/unsubstituted ring.

In some embodiments, R₁ at each occurrence is independently selected from a cyano group, a nitro group, a nitroso group, —CF₃, —Cl, —B, —I, —F, or a cyano-, nitro-, nitroso-, CF₃—, Cl—, B—, I—, or F-substituted aromatic or heteroaromatic group.

In some embodiments, at least one of X₁, X₂, Z₁, Z₂, Z₃, or Z₄ comprises a cyano group. In some embodiments, at least two of X₁, X₂, Z₁, Z₂, Z₃, or Z₄ comprises a cyano group. In some embodiments, at least three of X₁, X₂, Z₁, Z₂, Z₃, or Z₄ comprises a cyano group. In some embodiments, at least four of X₁, X₂, Z₁, Z₂, Z₃, or Z₄ comprises a cyano group. In some embodiments, at least five of X₁, X₂, Z₁, Z₂, Z₃, or Z₄ comprises a cyano group. In some embodiments, all of X₁, X₂, Z₁, Z₂, Z₃, and Z₄ comprise a cyano group.

In some embodiments, at least one of X₁ or X₂ comprises a cyano group. Preferably, both X₁ and X₂ comprise a cyano group.

In some embodiments, the organic compound comprises the substructure (I-1a) of the following formula (I-1), the LUMO thereof ≤−2.8 eV, preferably ≤−2.9 eV, more preferably ≤−3.0 eV, and most preferably ≤−3.1 eV.

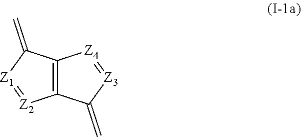

In some embodiments, the organic compound comprises a structure of one of formulas (II-1) to (II-3):

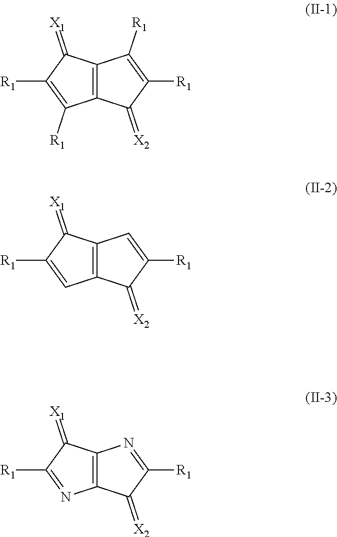

Where R₁, X₁, X₂ are identically defined as described above.

In some embodiments, in the organic compound as described herein, each of X₁ and X₂ at each occurrence is independently selected from CR₂R₃ or NR₂.

In some embodiments, in the organic compound as described herein, each of X₁ and X₂ at each occurrence is independently selected from CR₂R₃. Preferably, R₂ and R₃ are independently selected from a cyano group, a nitro group, a nitroso group, —CF₃, —Cl, —B, —I, —F, or a cyano-, nitro-, nitroso-, CF₃—, Cl—, B—, I—, or F-substituted aromatic or heteroaromatic group.

In some embodiments, each of X₁ and X₂ is independently selected from the following structural groups:

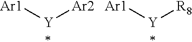

Where Y is C or Si, R₈ is defined as the above-mentioned R₁, Ar1 and Ar2 are independently selected from a substituted/unsubstituted aromatic or heteroaromatic group containing 5 to 60 ring atoms, the LUMO of Ar1 and/or Ar2≤−2.9 eV, preferably ≤−3.0 eV, more preferably ≤−3.1 eV, and most preferably ≤−3.2 eV.

In some embodiments, each of X₁ and X₂ is independently selected from the following groups:

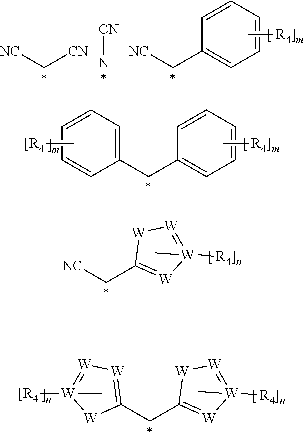

Where R₄ at each occurrence is independently selected from a cyano group, a nitro group, a nitroso group, —CF₃, —Cl, —B, —I, —F, or —OCF₃; m is an integer from 0 to 5; n is an integer from 0 to 4; W at each occurrence is independently selected from O, S, or N; * denotes a linkage site.

Preferably, m is an integer from 1, 2, 3, 4, 5; n is an integer from 1, 2, 3, 4.

In some embodiments, each of X₁ and X₂ is independently selected from the following groups:

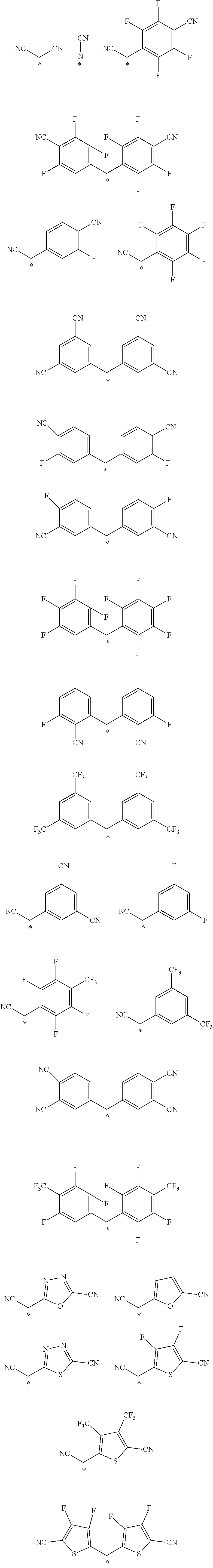

Where * denotes a linkage site.

In some embodiments, R₁ is independently selected from a cyano group, a nitro group, a nitroso group, —CF₃, —Cl, —B, —I, —F, or —OCF₃, or a cyano-, nitro-, nitroso-, CF₃—, Cl—, B—, I—, F—, or OCF₃-substituted aromatic or heteroaromatic group.

Where the cyano-, nitro-, nitroso-, CF₃—, Cl—, B—, I—, F—, or OCF₃-substituted aromatic or heteroaromatic group is preferably selected from the following groups:

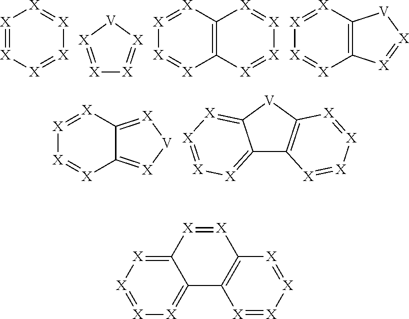

Where each X at each occurrence is independently selected, from CR₅ or N; each V at each occurrence is independently selected from CR₆R₇, NR₆, O, S, SiR₆R₇, PR₆, P(═O)R₆, S═O, S(═O)₂, or C═O; each of R₅ to R₇ at each occurrence is independently selected, from —H, -D, a C₁-C₂₀ linear alkyl group, a C₃-C₂₀ branched/cyclic alkyl group, a C₁-C₂₀ ketone group, a C₂-C₂₀ alkoxycarbonyl group, a C₇-C₂₀ aryloxycarbonyl group, a cyano group, a carbamoyl group, a nitro group, a nitroso group, —CF₃, —Cl, —B, —I, —F, a substituted/unsubstituted aromatic or heteroaromatic group containing 5 to 60 ring atoms, an aryloxy or heteroaryloxy group containing 5 to 60 ring atoms, or any combination thereof; and at least one R₇ is selected from a cyano group, a nitro group, a nitroso group, —CF₃, —Cl, —B, —I, —F, or —OCF₃.

Furtherly, the cyano-, nitro-, nitroso-, CF₃—, Cl—, B—, I—, F—, or OCF₃-substituted aromatic or heteroaromatic group is preferably selected from the following groups:

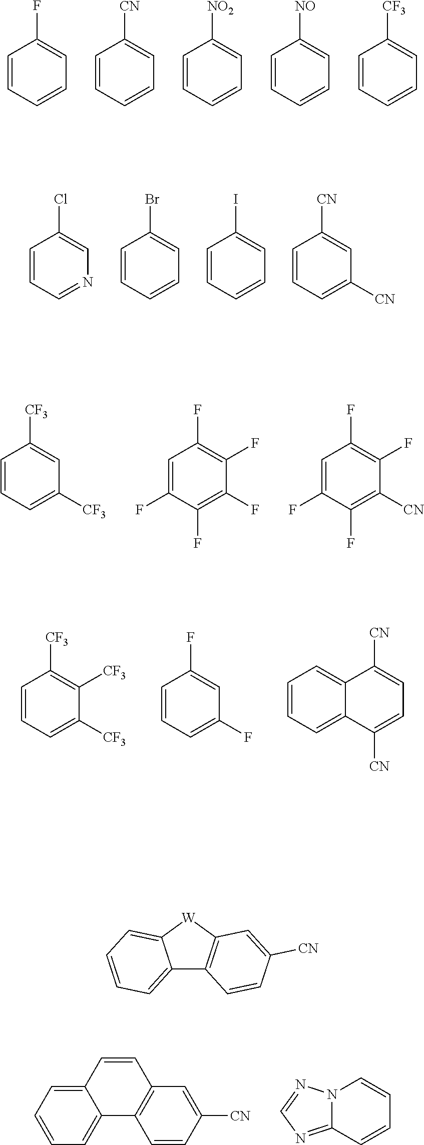

Specific examples of the organic compounds of formula (I-1) as described herein are listed below, but not limited thereto.

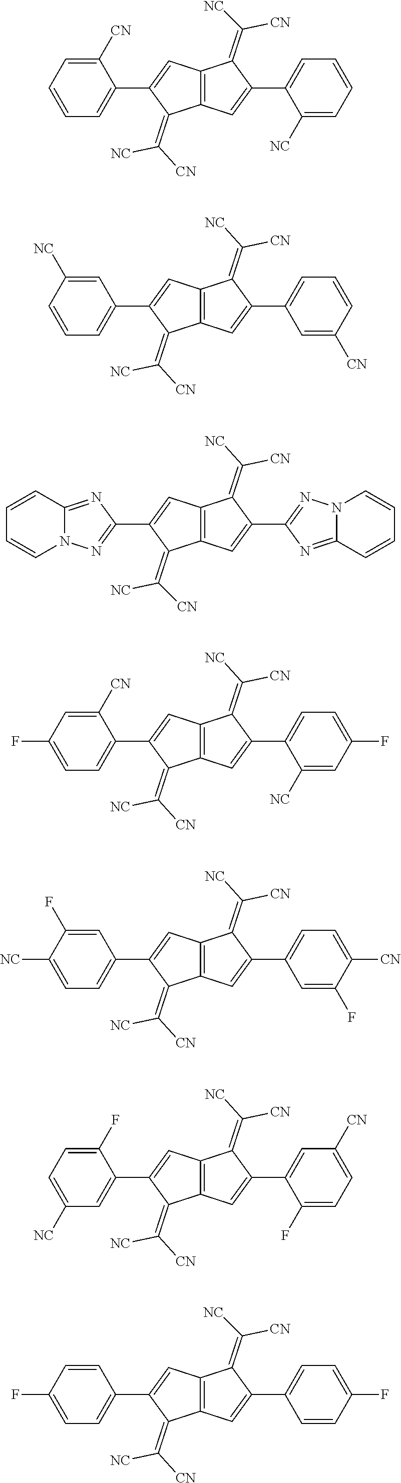

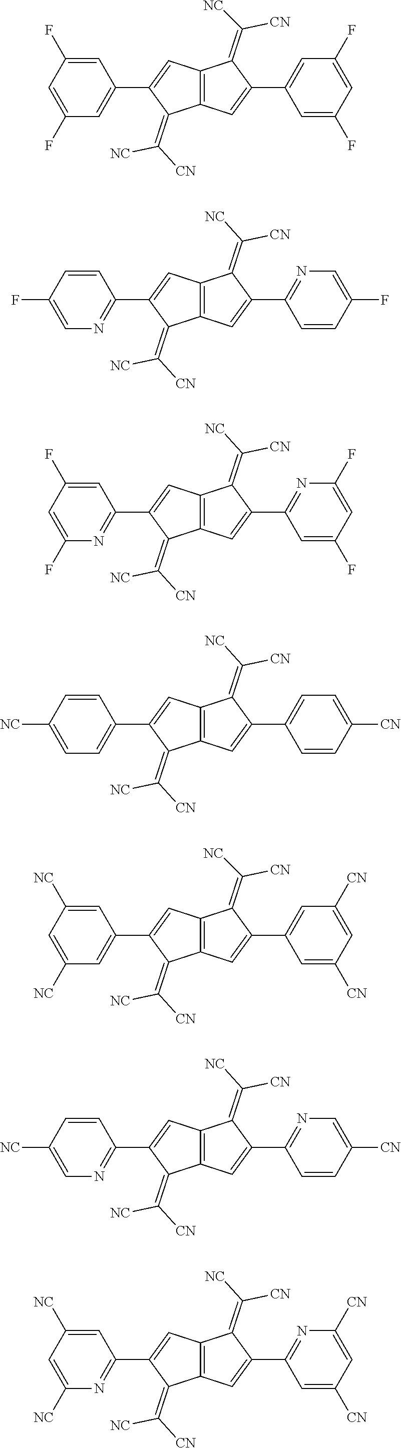

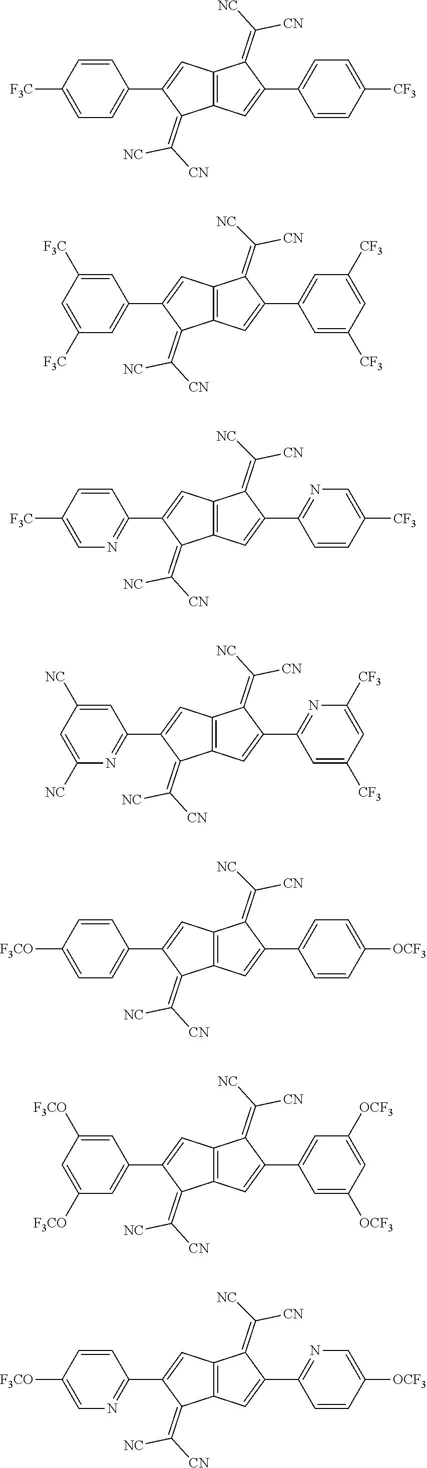

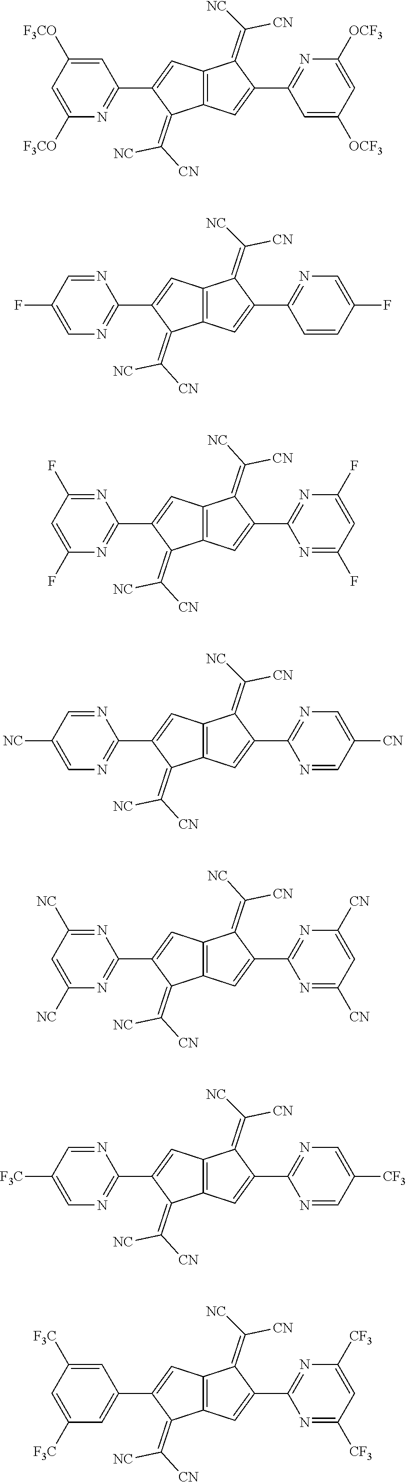

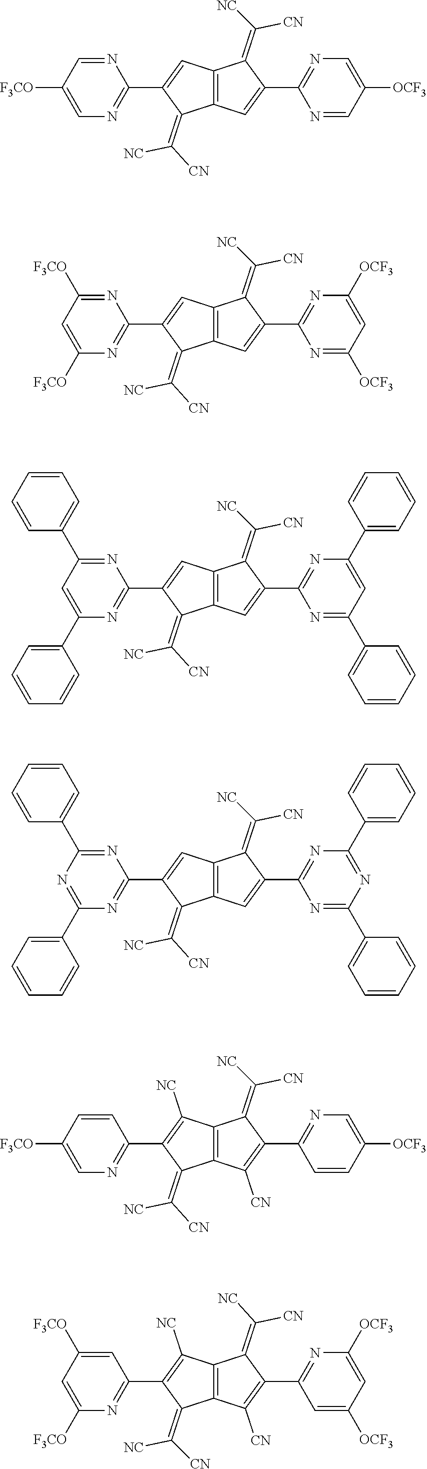

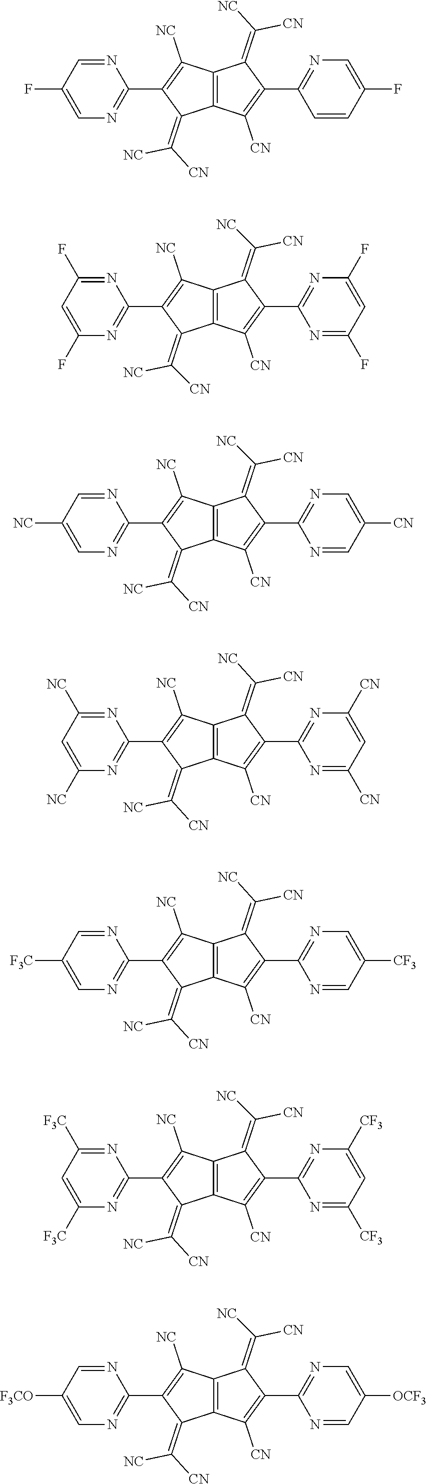

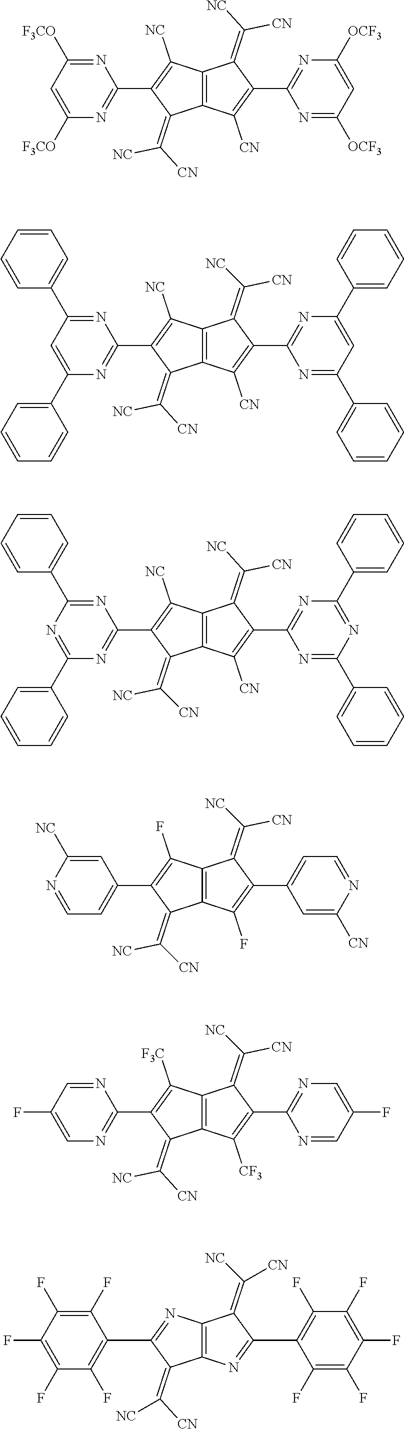

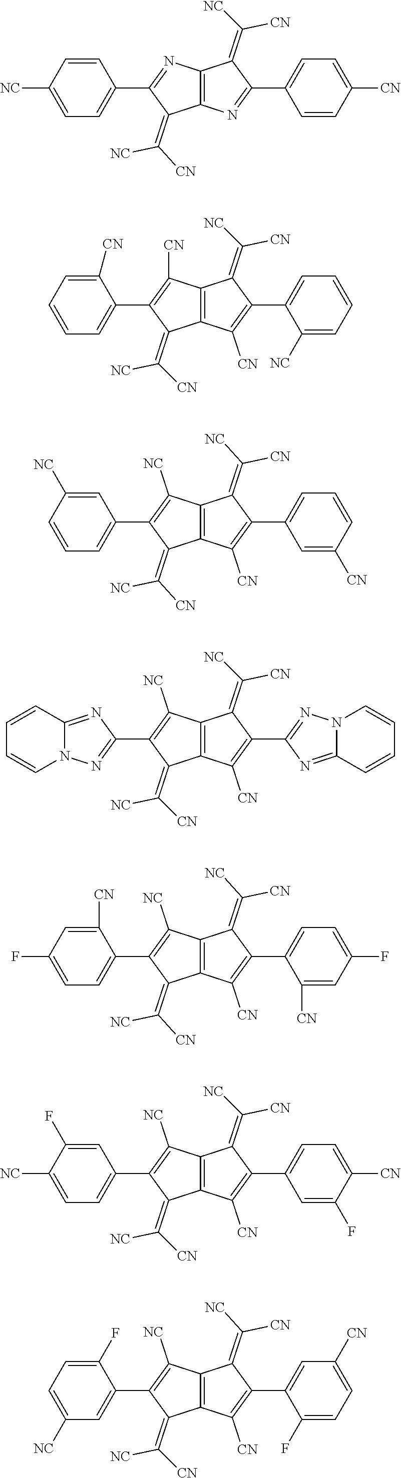

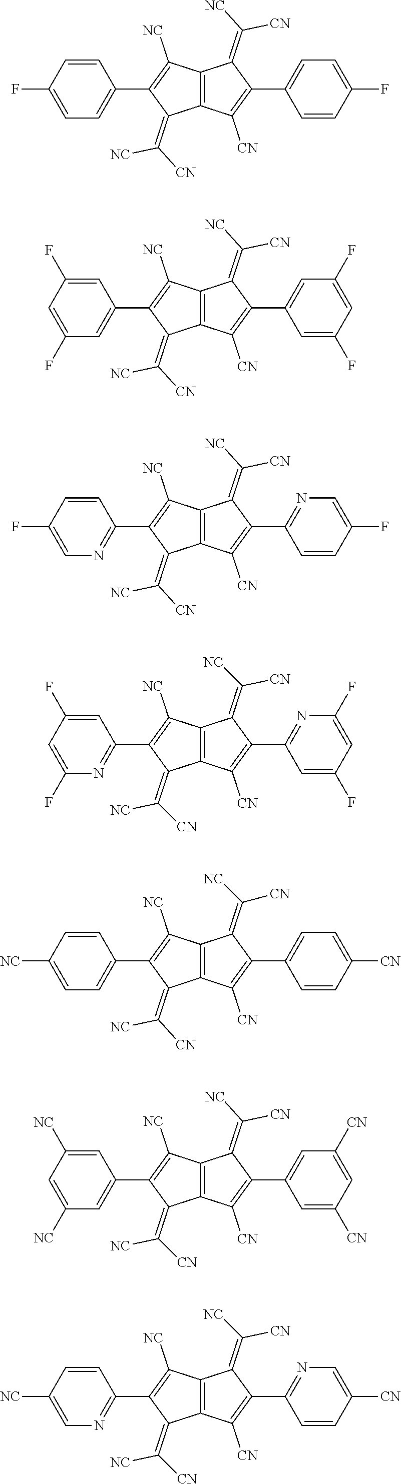

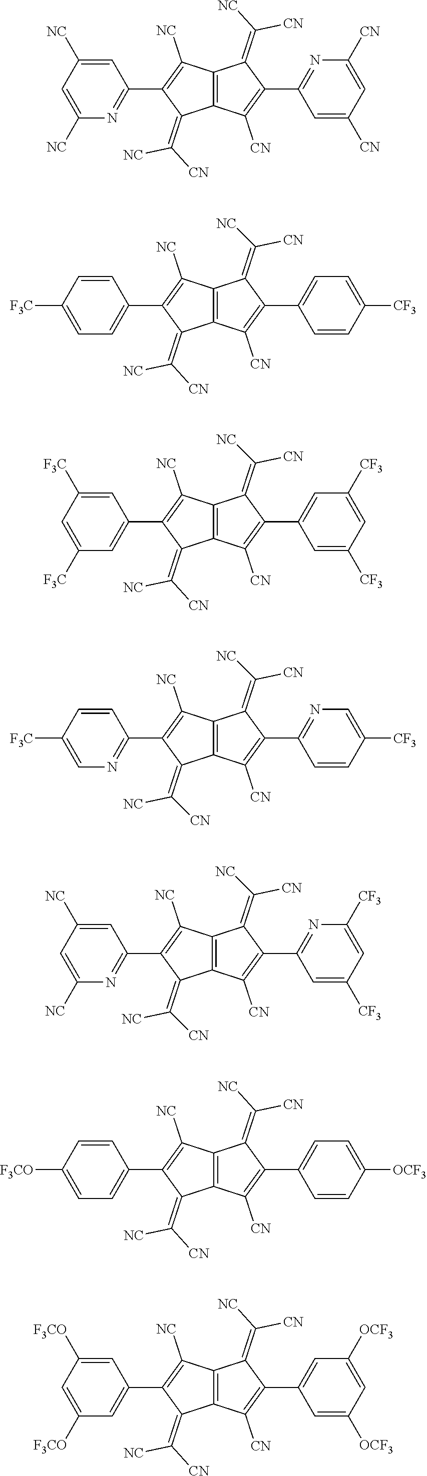

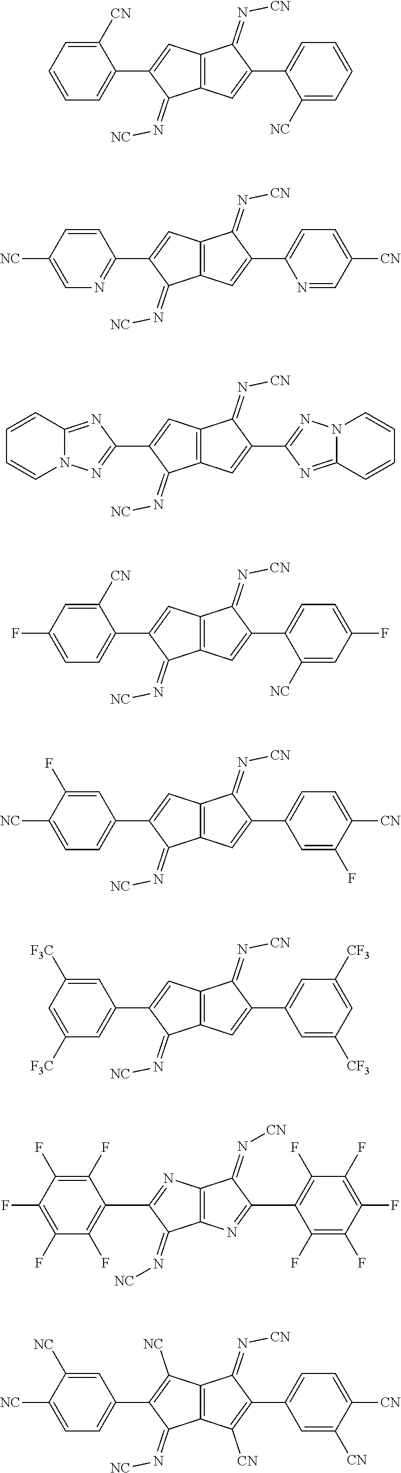

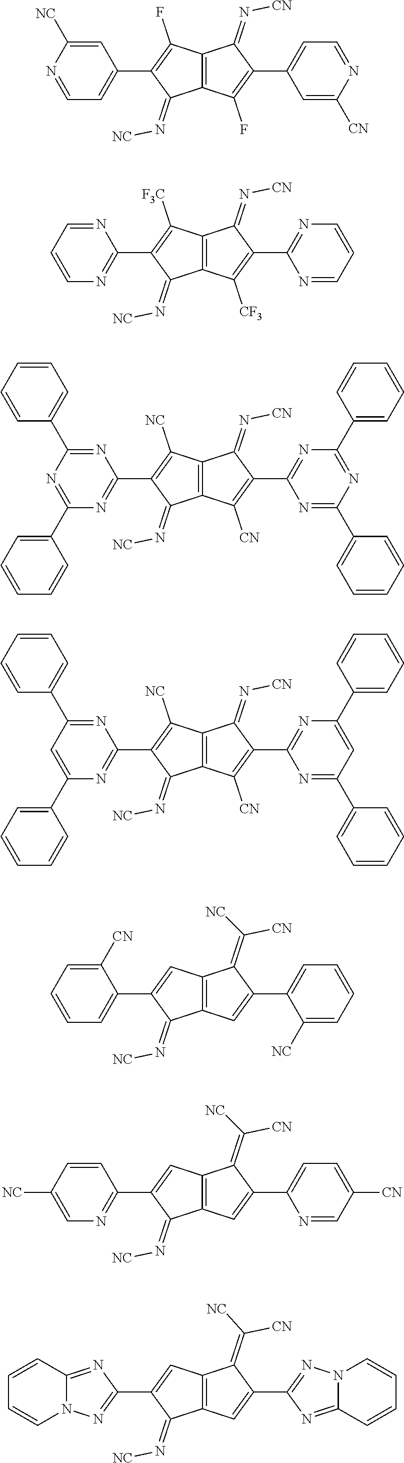

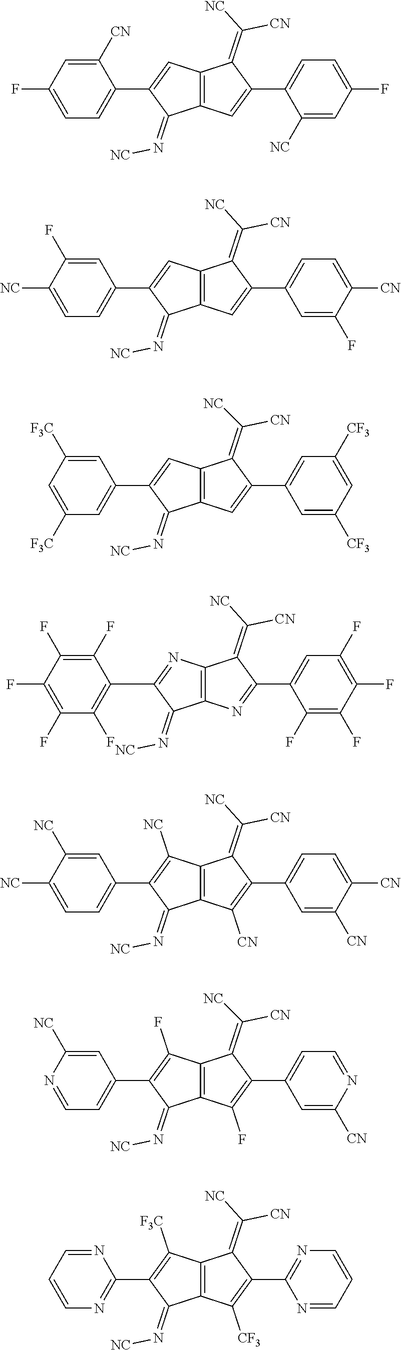

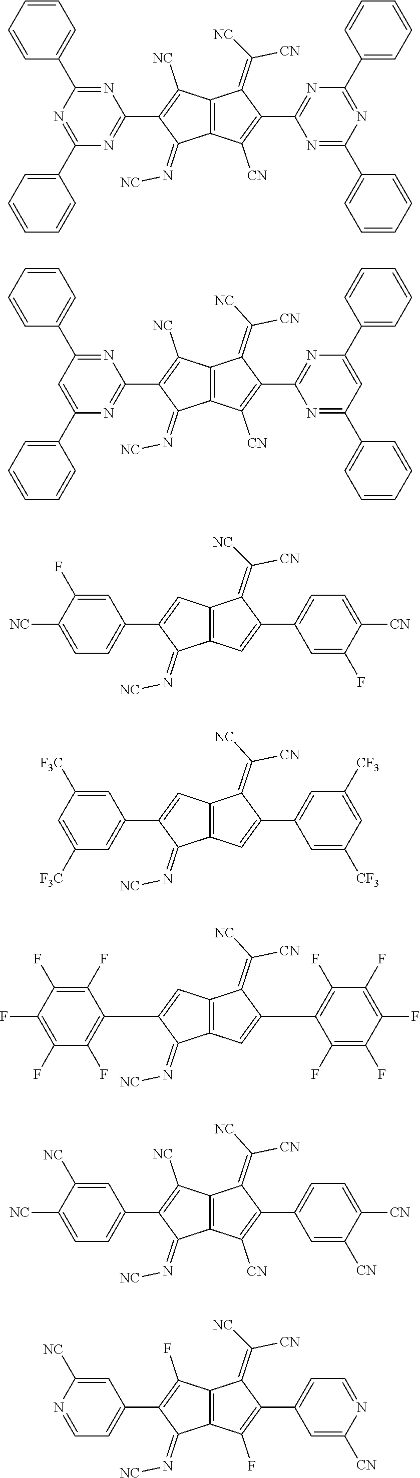

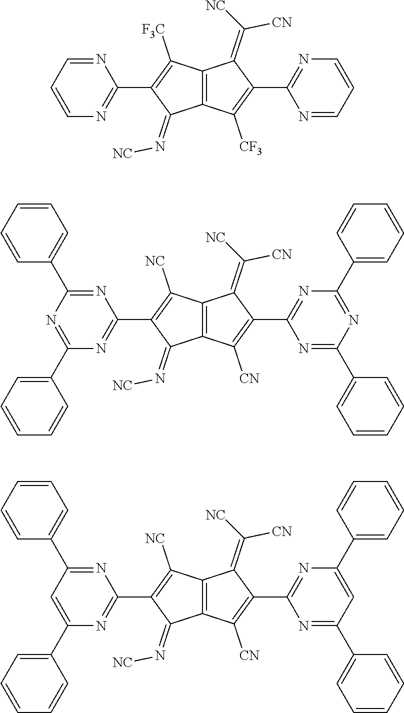

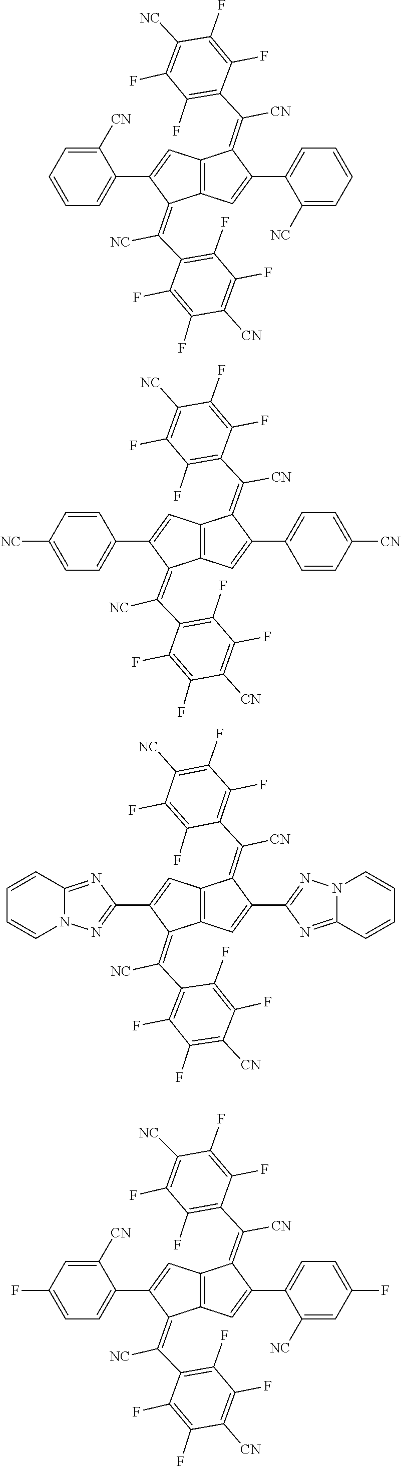

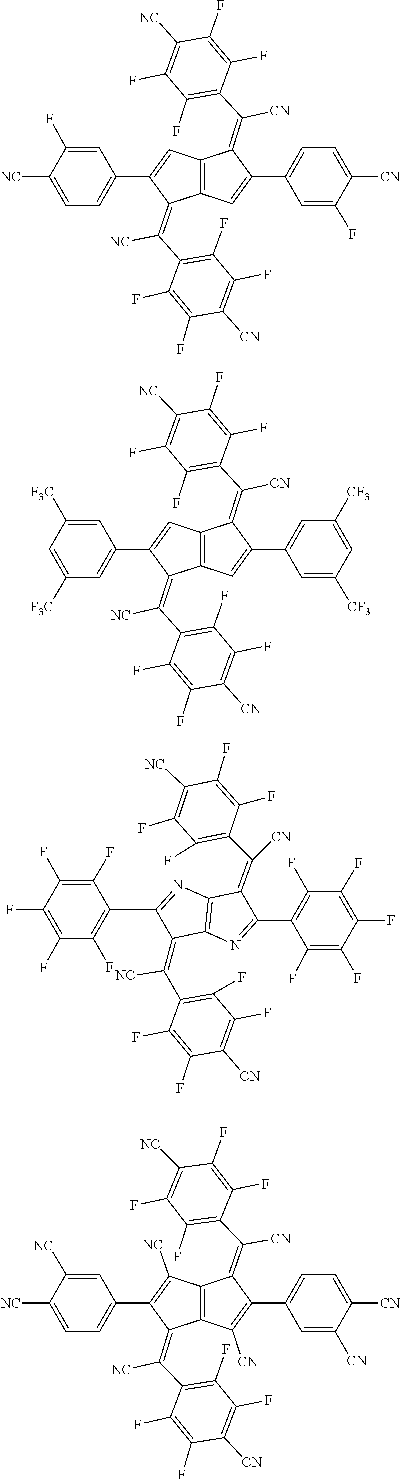

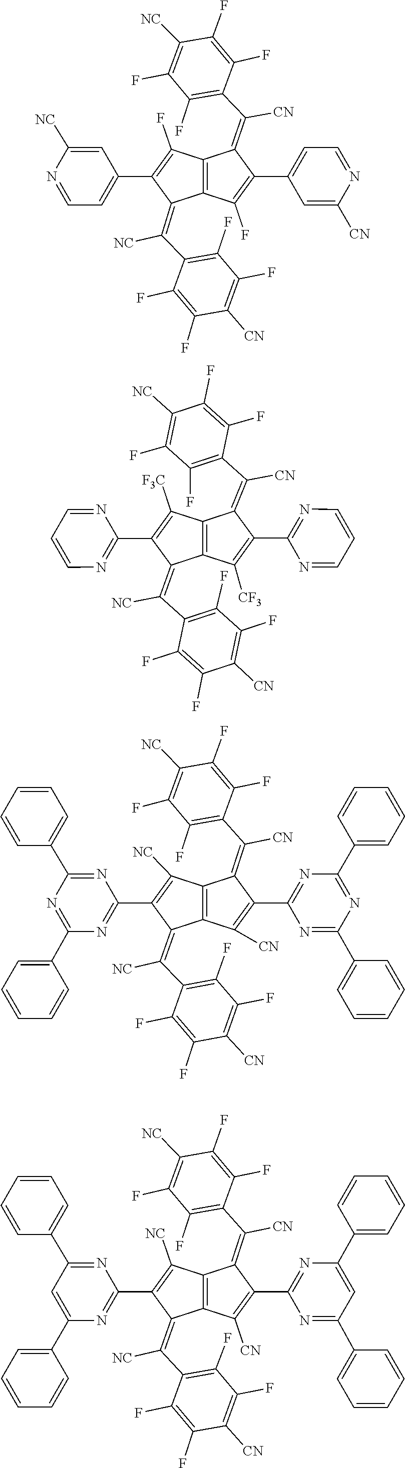

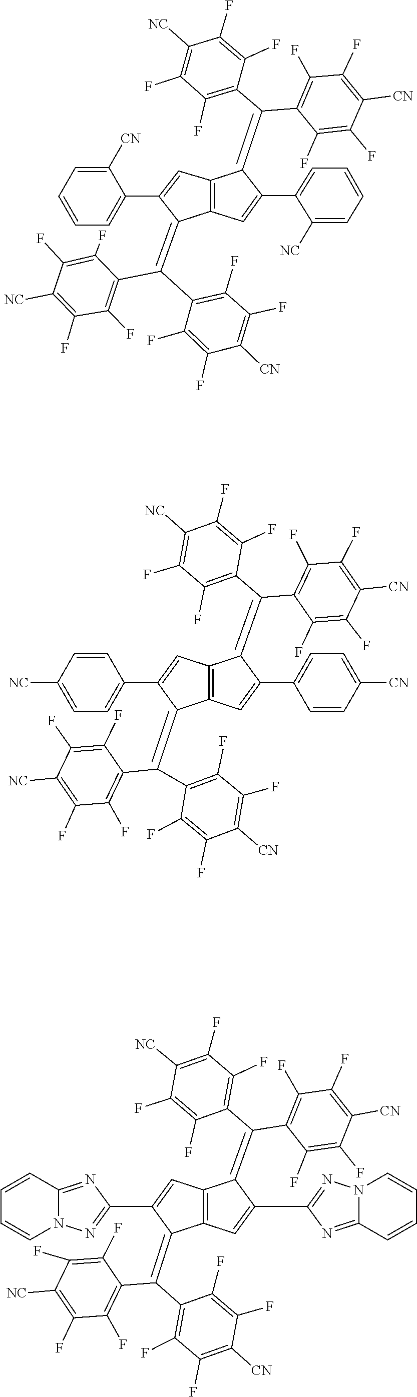 [SMILES: N#Cc1ccccc1C1=CC2=C(C=C(c3ccccc3C#N)C2=C(c2c(F)c(F)c(C#N)c(F)c2F)c2c(F)c(F)c(C#N)c(F)c2F)C1=C(c1c(F)c(F)c(C#N)c(F)c1F)c1c(F)c(F)c(C#N)c(F)c1F ; N#Cc1ccc(C2=CC3=C(C=C(c4ccc(C#N)cc4)C3=C(c3c(F)c(F)c(C#N)c(F)c3F)c3c(F)c(F)c(C#N)c(F)c3F)C2=C(c2c(F)c(F)c(C#N)c(F)c2F)c2c(F)c(F)c(C#N)c(F)c2F)cc1 ; N#Cc1c(F)c(F)c(C(=C2C(c3nc4ccccn4n3)=CC3=C2C=C(c2nc4ccccn4n2)C3=C(c2c(F)c(F)c(C#N)c(F)c2F)c2c(F)c(F)c(C#N)c(F)c2F)c2c(F)c(F)c(C#N)c(F)c2F)c(F)c1F]

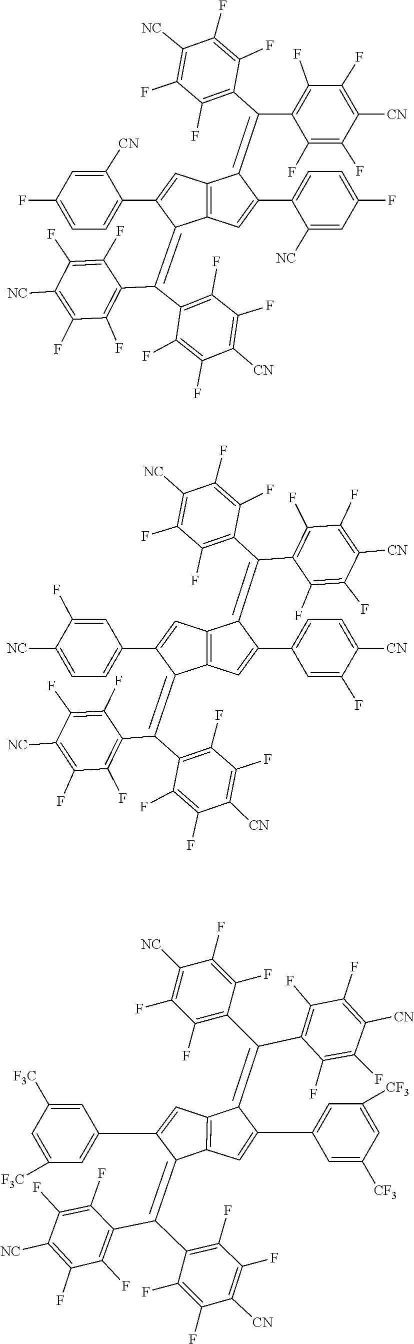 [SMILES: N#Cc1cc(F)ccc1C1=CC2=C(C=C(c3ccc(F)cc3C#N)C2=C(c2c(F)c(F)c(C#N)c(F)c2F)c2c(F)c(F)c(C#N)c(F)c2F)C1=C(c1c(F)c(F)c(C#N)c(F)c1F)c1c(F)c(F)c(C#N)c(F)c1F ; N#Cc1ccc(C2=CC3=C(C=C(c4ccc(C#N)c(F)c4)C3=C(c3c(F)c(F)c(C#N)c(F)c3F)c3c(F)c(F)c(C#N)c(F)c3F)C2=C(c2c(F)c(F)c(C#N)c(F)c2F)c2c(F)c(F)c(C#N)c(F)c2F)cc1F ; N#Cc1c(F)c(F)c(C(=C2C(c3cc(C(F)(F)F)cc(C(F)(F)F)c3)=CC3=C2C=C(c2cc(C(F)(F)F)cc(C(F)(F)F)c2)C3=C(c2c(F)c(F)c(C#N)c(F)c2F)c2c(F)c(F)c(C#N)c(F)c2F)c2c(F)c(F)c(C#N)c(F)c2F)c(F)c1F]

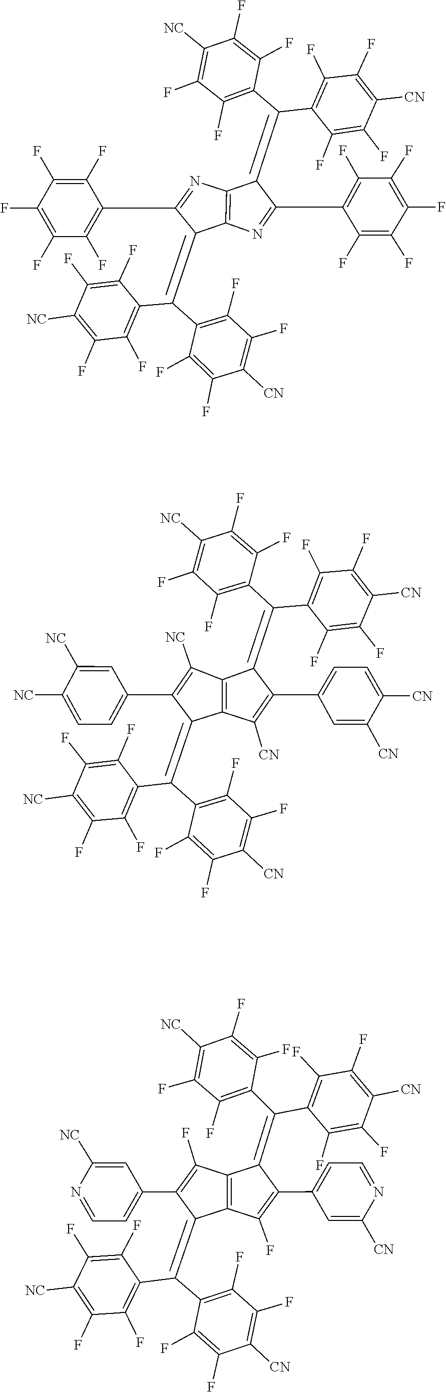 [SMILES: N#Cc1c(F)c(F)c(C(=C2C(c3c(F)c(F)c(F)c(F)c3F)=NC3=C2N=C(c2c(F)c(F)c(F)c(F)c2F)C3=C(c2c(F)c(F)c(C#N)c(F)c2F)c2c(F)c(F)c(C#N)c(F)c2F)c2c(F)c(F)c(C#N)c(F)c2F)c(F)c1F ; N#CC1=C(c2ccc(C#N)c(C#N)c2)C(=C(c2c(F)c(F)c(C#N)c(F)c2F)c2c(F)c(F)c(C#N)c(F)c2F)C2=C1C(=C(c1c(F)c(F)c(C#N)c(F)c1F)c1c(F)c(F)c(C#N)c(F)c1F)C(c1ccc(C#N)c(C#N)c1)=C2C#N ; N#Cc1cc(C2=C(F)C3=C(C(F)=C(c4ccnc(C#N)c4)C3=C(c3c(F)c(F)c(C#N)c(F)c3F)c3c(F)c(F)c(C#N)c(F)c3F)C2=C(c2c(F)c(F)c(C#N)c(F)c2F)c2c(F)c(F)c(C#N)c(F)c2F)ccn1]

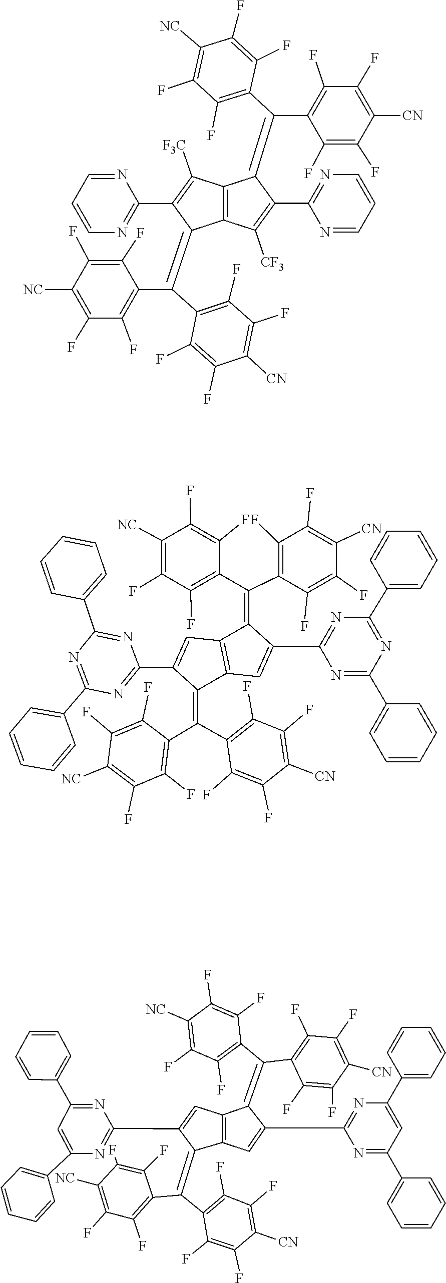

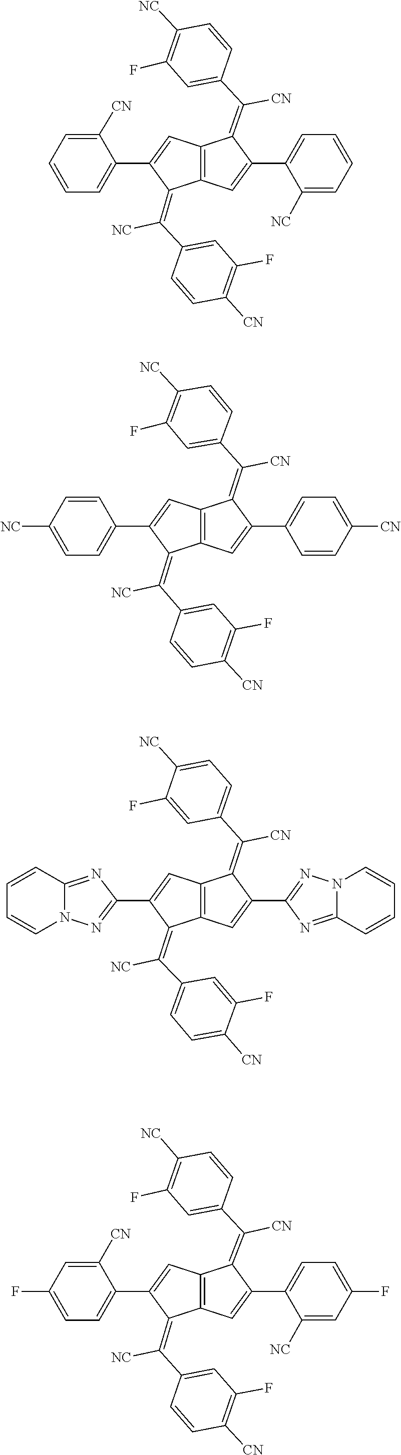 [SMILES: N#C/C(=C1\C(c2ccccc2C#N)=CC2=C1C=C(c1ccccc1C#N)/C2=C(\C#N)c1ccc(C#N)c(F)c1)c1ccc(C#N)c(F)c1 ; N#C/C(=C1\C(c2ccc(C#N)cc2)=CC2=C1C=C(c1ccc(C#N)cc1)/C2=C(\C#N)c1ccc(C#N)c(F)c1)c1ccc(C#N)c(F)c1 ; N#C/C(=C1\C(c2nc3ccccn3n2)=CC2=C1C=C(c1nc3ccccn3n1)/C2=C(\C#N)c1ccc(C#N)c(F)c1)c1ccc(C#N)c(F)c1 ; N#C/C(=C1\C(c2ccc(F)cc2C#N)=CC2=C1C=C(c1ccc(F)cc1C#N)/C2=C(\C#N)c1ccc(C#N)c(F)c1)c1ccc(C#N)c(F)c1]

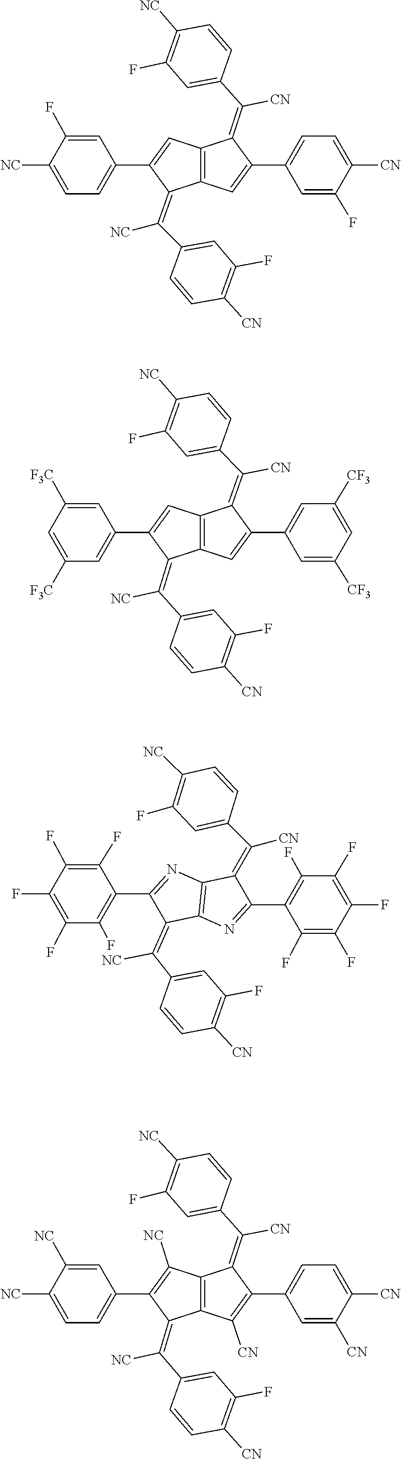 [SMILES: N#C/C(=C1\C(c2ccc(C#N)c(F)c2)=CC2=C1C=C(c1ccc(C#N)c(F)c1)/C2=C(\C#N)c1ccc(C#N)c(F)c1)c1ccc(C#N)c(F)c1 ; N#C/C(=C1\C(c2cc(C(F)(F)F)cc(C(F)(F)F)c2)=CC2=C1C=C(c1cc(C(F)(F)F)cc(C(F)(F)F)c1)/C2=C(\C#N)c1ccc(C#N)c(F)c1)c1ccc(C#N)c(F)c1 ; N#C/C(=C1\C(c2c(F)c(F)c(F)c(F)c2F)=NC2=C1N=C(c1c(F)c(F)c(F)c(F)c1F)/C2=C(\C#N)c1ccc(C#N)c(F)c1)c1ccc(C#N)c(F)c1 ; N#CC1=C(c2ccc(C#N)c(C#N)c2)/C(=C(\C#N)c2ccc(C#N)c(F)c2)C2=C1/C(=C(/C#N)c1ccc(C#N)c(F)c1)C(c1ccc(C#N)c(C#N)c1)=C2C#N]

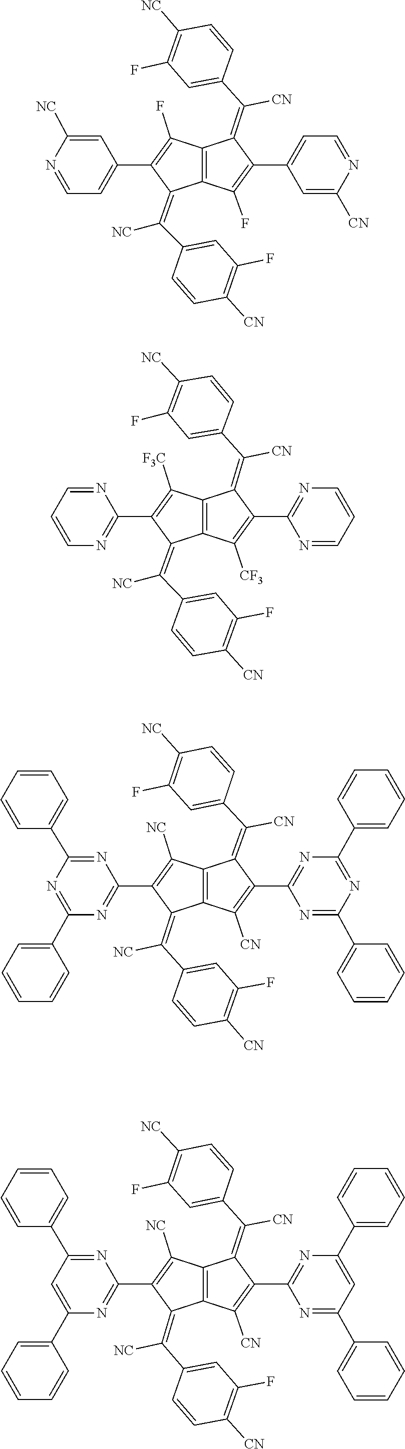 [SMILES: N#C/C(=C1C2=C(C(F)=C/1c1ccnc(C#N)c1)/C(=C(/C#N)c1ccc(C#N)c(F)c1)C(c1ccnc(C#N)c1)=C2F)c1ccc(C#N)c(F)c1 ; N#C/C(=C1C2=C(C(C(F)(F)F)=C/1c1ncccn1)/C(=C(/C#N)c1ccc(C#N)c(F)c1)C(c1ncccn1)=C2C(F)(F)F)c1ccc(C#N)c(F)c1 ; N#CC1=C(c2nc(-c3ccccc3)nc(-c3ccccc3)n2)/C(=C(\C#N)c2ccc(C#N)c(F)c2)C2=C1/C(=C(/C#N)c1ccc(C#N)c(F)c1)C(c1nc(-c3ccccc3)nc(-c3ccccc3)n1)=C2C#N ; N#CC1=C(c2nc(-c3ccccc3)cc(-c3ccccc3)n2)/C(=C(\C#N)c2ccc(C#N)c(F)c2)C2=C1/C(=C(/C#N)c1ccc(C#N)c(F)c1)C(c1nc(-c3ccccc3)cc(-c3ccccc3)n1)=C2C#N]

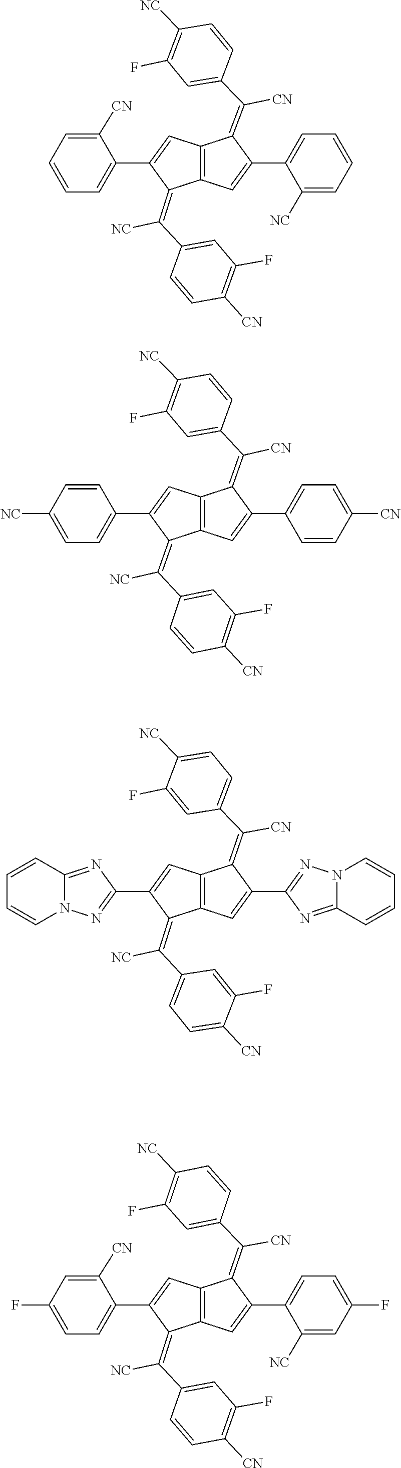 [SMILES: N#C/C(=C1\C(c2ccccc2C#N)=CC2=C1C=C(c1ccccc1C#N)/C2=C(\C#N)c1ccc(C#N)c(F)c1)c1ccc(C#N)c(F)c1 ; N#C/C(=C1\C(c2ccc(C#N)cc2)=CC2=C1C=C(c1ccc(C#N)cc1)/C2=C(\C#N)c1ccc(C#N)c(F)c1)c1ccc(C#N)c(F)c1 ; N#C/C(=C1\C(c2nc3ccccn3n2)=CC2=C1C=C(c1nc3ccccn3n1)/C2=C(\C#N)c1ccc(C#N)c(F)c1)c1ccc(C#N)c(F)c1 ; N#C/C(=C1\C(c2ccc(F)cc2C#N)=CC2=C1C=C(c1ccc(F)cc1C#N)/C2=C(\C#N)c1ccc(C#N)c(F)c1)c1ccc(C#N)c(F)c1]

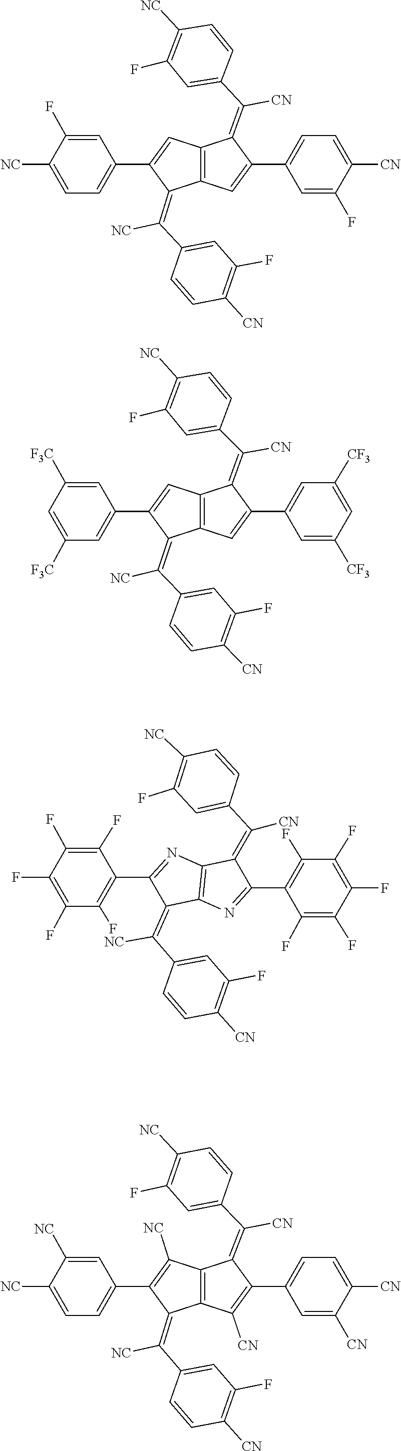 [SMILES: N#C/C(=C1\C(c2ccc(C#N)c(F)c2)=CC2=C1C=C(c1ccc(C#N)c(F)c1)/C2=C(\C#N)c1ccc(C#N)c(F)c1)c1ccc(C#N)c(F)c1 ; N#C/C(=C1\C(c2cc(C(F)(F)F)cc(C(F)(F)F)c2)=CC2=C1C=C(c1cc(C(F)(F)F)cc(C(F)(F)F)c1)/C2=C(\C#N)c1ccc(C#N)c(F)c1)c1ccc(C#N)c(F)c1 ; N#C/C(=C1\C(c2c(F)c(F)c(F)c(F)c2F)=NC2=C1N=C(c1c(F)c(F)c(F)c(F)c1F)/C2=C(\C#N)c1ccc(C#N)c(F)c1)c1ccc(C#N)c(F)c1 ; N#CC1=C(c2ccc(C#N)c(C#N)c2)/C(=C(\C#N)c2ccc(C#N)c(F)c2)C2=C1/C(=C(/C#N)c1ccc(C#N)c(F)c1)C(c1ccc(C#N)c(C#N)c1)=C2C#N]

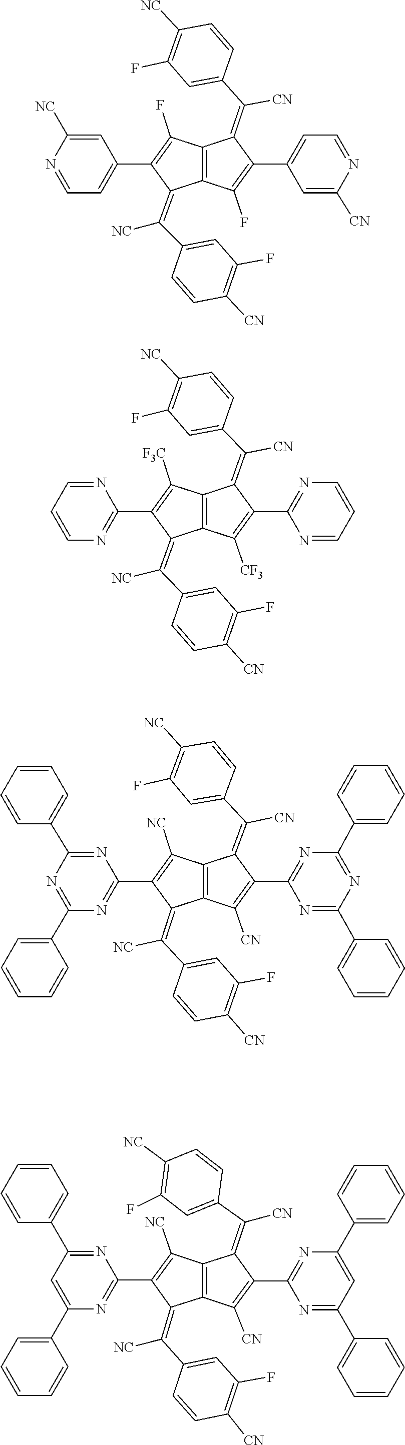 [SMILES: N#C/C(=C1C2=C(C(F)=C/1c1ccnc(C#N)c1)/C(=C(/C#N)c1ccc(C#N)c(F)c1)C(c1ccnc(C#N)c1)=C2F)c1ccc(C#N)c(F)c1 ; N#C/C(=C1C2=C(C(C(F)(F)F)=C/1c1ncccn1)/C(=C(/C#N)c1ccc(C#N)c(F)c1)C(c1ncccn1)=C2C(F)(F)F)c1ccc(C#N)c(F)c1 ; N#CC1=C(c2nc(-c3ccccc3)nc(-c3ccccc3)n2)/C(=C(\C#N)c2ccc(C#N)c(F)c2)C2=C1/C(=C(/C#N)c1ccc(C#N)c(F)c1)C(c1nc(-c3ccccc3)nc(-c3ccccc3)n1)=C2C#N ; N#CC1=C(c2nc(-c3ccccc3)cc(-c3ccccc3)n2)/C(=C(\C#N)c2ccc(C#N)c(F)c2)C2=C1/C(=C(/C#N)c1ccc(C#N)c(F)c1)C(c1nc(-c3ccccc3)cc(-c3ccccc3)n1)=C2C#N]

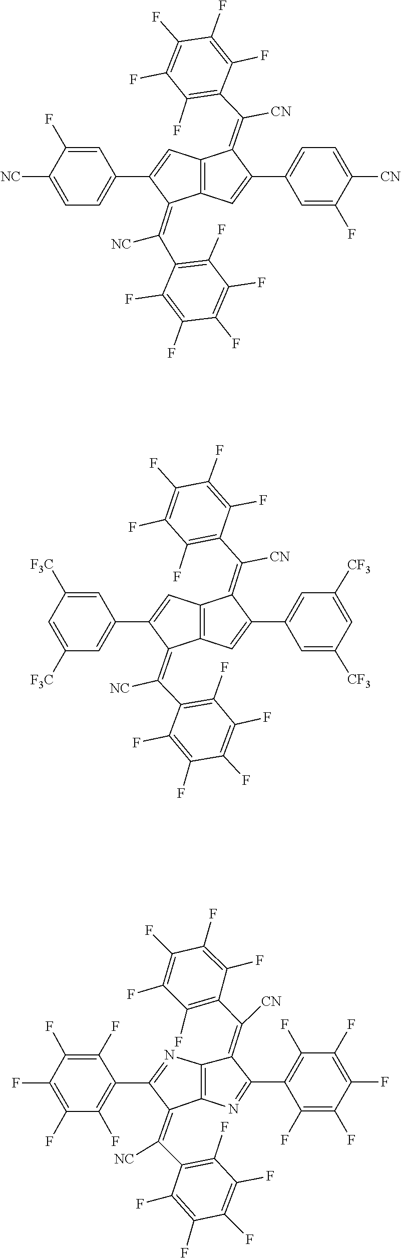 [SMILES: N#C/C(=C1\C(c2ccc(C#N)c(F)c2)=CC2=C1C=C(c1ccc(C#N)c(F)c1)/C2=C(\C#N)c1c(F)c(F)c(F)c(F)c1F)c1c(F)c(F)c(F)c(F)c1F ; N#C/C(=C1\C(c2cc(C(F)(F)F)cc(C(F)(F)F)c2)=CC2=C1C=C(c1cc(C(F)(F)F)cc(C(F)(F)F)c1)/C2=C(\C#N)c1c(F)c(F)c(F)c(F)c1F)c1c(F)c(F)c(F)c(F)c1F ; N#C/C(=C1\C(c2c(F)c(F)c(F)c(F)c2F)=NC2=C1N=C(c1c(F)c(F)c(F)c(F)c1F)/C2=C(\C#N)c1c(F)c(F)c(F)c(F)c1F)c1c(F)c(F)c(F)c(F)c1F]

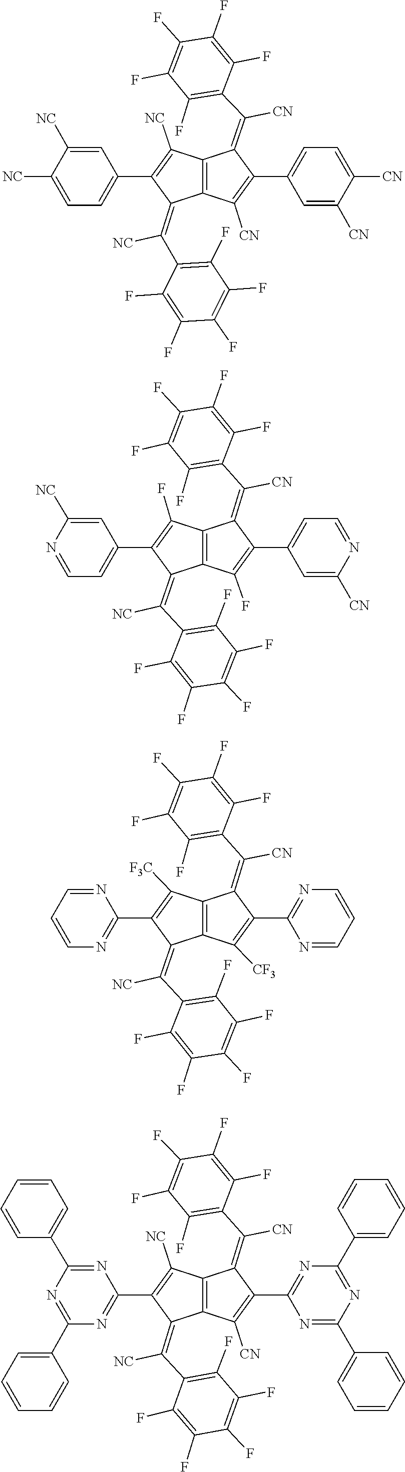

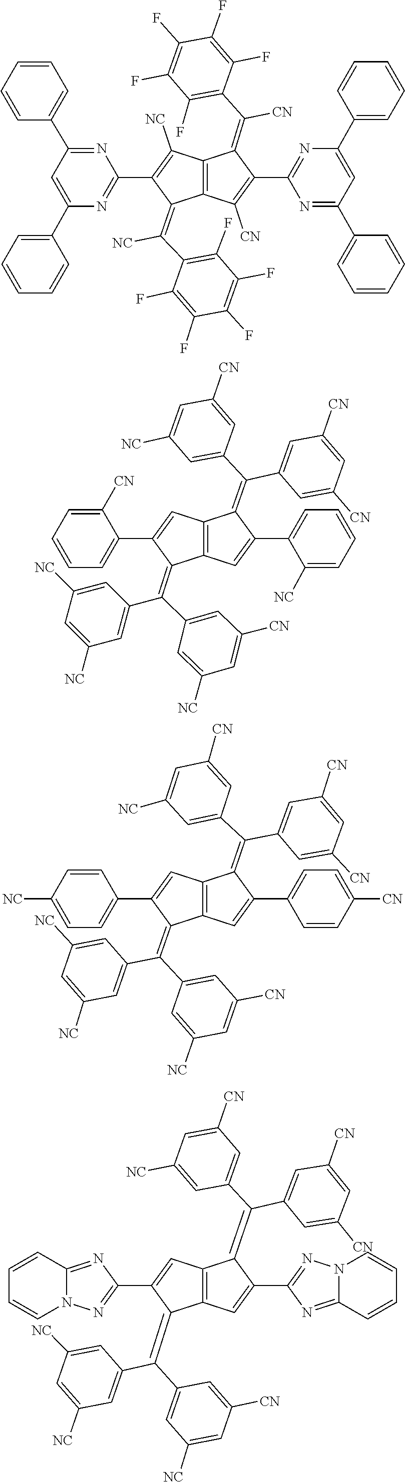

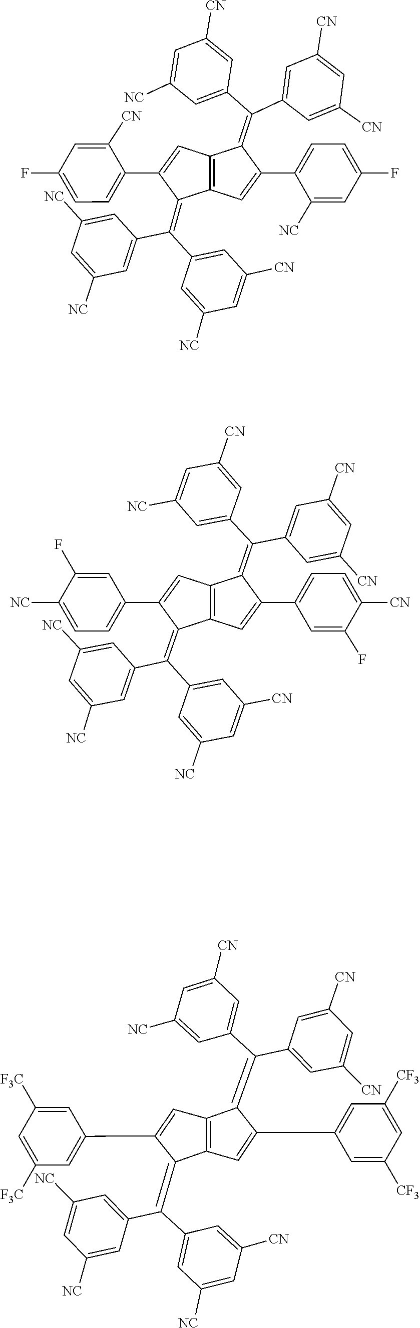 [SMILES: N#Cc1cc(C#N)cc(C(=C2C(c3ccc(F)cc3C#N)=CC3=C2C=C(c2ccc(F)cc2C#N)C3=C(c2cc(C#N)cc(C#N)c2)c2cc(C#N)cc(C#N)c2)c2cc(C#N)cc(C#N)c2)c1 ; N#Cc1cc(C#N)cc(C(=C2C(c3ccc(C#N)c(F)c3)=CC3=C2C=C(c2ccc(C#N)c(F)c2)C3=C(c2cc(C#N)cc(C#N)c2)c2cc(C#N)cc(C#N)c2)c2cc(C#N)cc(C#N)c2)c1 ; N#Cc1cc(C#N)cc(C(=C2C(c3cc(C(F)(F)F)cc(C(F)(F)F)c3)=CC3=C2C=C(c2cc(C(F)(F)F)cc(C(F)(F)F)c2)C3=C(c2cc(C#N)cc(C#N)c2)c2cc(C#N)cc(C#N)c2)c2cc(C#N)cc(C#N)c2)c1]

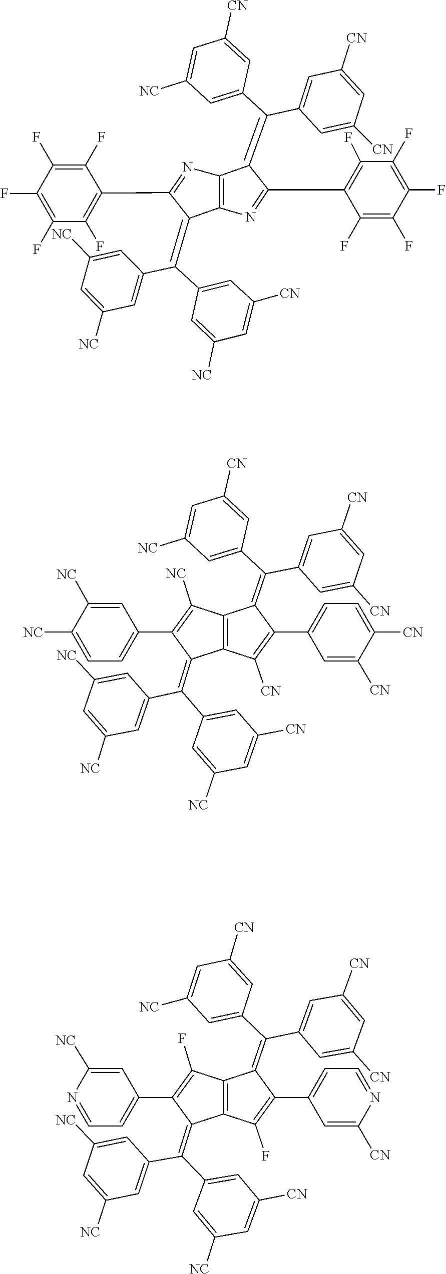 [SMILES: N#Cc1cc(C#N)cc(C(=C2C(c3c(F)c(F)c(F)c(F)c3F)=NC3=C2N=C(c2c(F)c(F)c(F)c(F)c2F)C3=C(c2cc(C#N)cc(C#N)c2)c2cc(C#N)cc(C#N)c2)c2cc(C#N)cc(C#N)c2)c1 ; N#CC1=C(c2ccc(C#N)c(C#N)c2)C(=C(c2cc(C#N)cc(C#N)c2)c2cc(C#N)cc(C#N)c2)C2=C1C(=C(c1cc(C#N)cc(C#N)c1)c1cc(C#N)cc(C#N)c1)C(c1ccc(C#N)c(C#N)c1)=C2C#N ; N#Cc1cc(C#N)cc(C(=C2C3=C(C(F)=C2c2ccnc(C#N)c2)C(=C(c2cc(C#N)cc(C#N)c2)c2cc(C#N)cc(C#N)c2)C(c2ccnc(C#N)c2)=C3F)c2cc(C#N)cc(C#N)c2)c1]

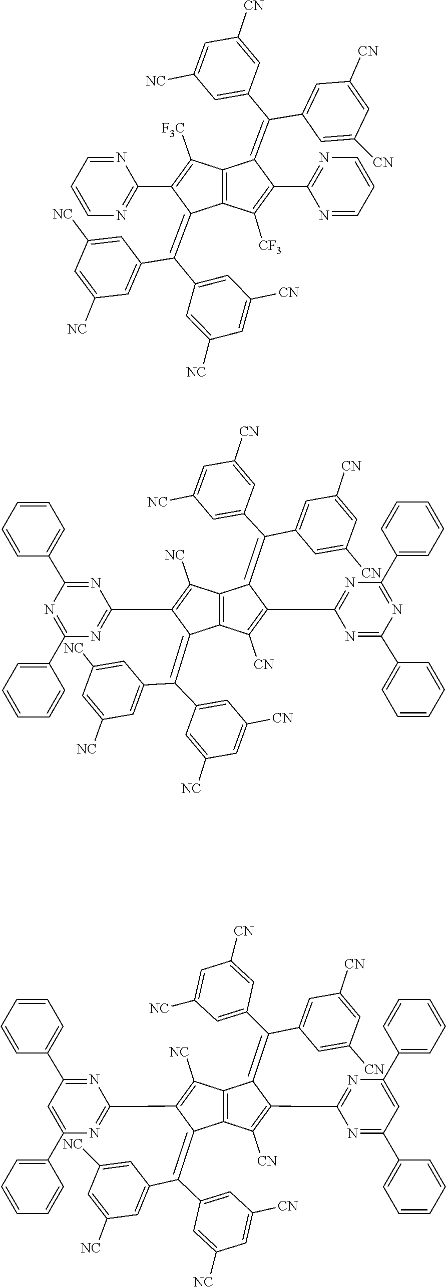 [SMILES: N#Cc1cc(C#N)cc(C(=C2C3=C(C(=C(c4cc(C#N)cc(C#N)c4)c4cc(C#N)cc(C#N)c4)C(c4ncccn4)=C3C(F)(F)F)C(C(F)(F)F)=C2c2ncccn2)c2cc(C#N)cc(C#N)c2)c1 ; N#CC1=C(c2nc(-c3ccccc3)nc(-c3ccccc3)n2)C(=C(c2cc(C#N)cc(C#N)c2)c2cc(C#N)cc(C#N)c2)C2=C1C(=C(c1cc(C#N)cc(C#N)c1)c1cc(C#N)cc(C#N)c1)C(c1nc(-c3ccccc3)nc(-c3ccccc3)n1)=C2C#N ; N#CC1=C(c2nc(-c3ccccc3)cc(-c3ccccc3)n2)C(=C(c2cc(C#N)cc(C#N)c2)c2cc(C#N)cc(C#N)c2)C2=C1C(=C(c1cc(C#N)cc(C#N)c1)c1cc(C#N)cc(C#N)c1)C(c1nc(-c3ccccc3)cc(-c3ccccc3)n1)=C2C#N]

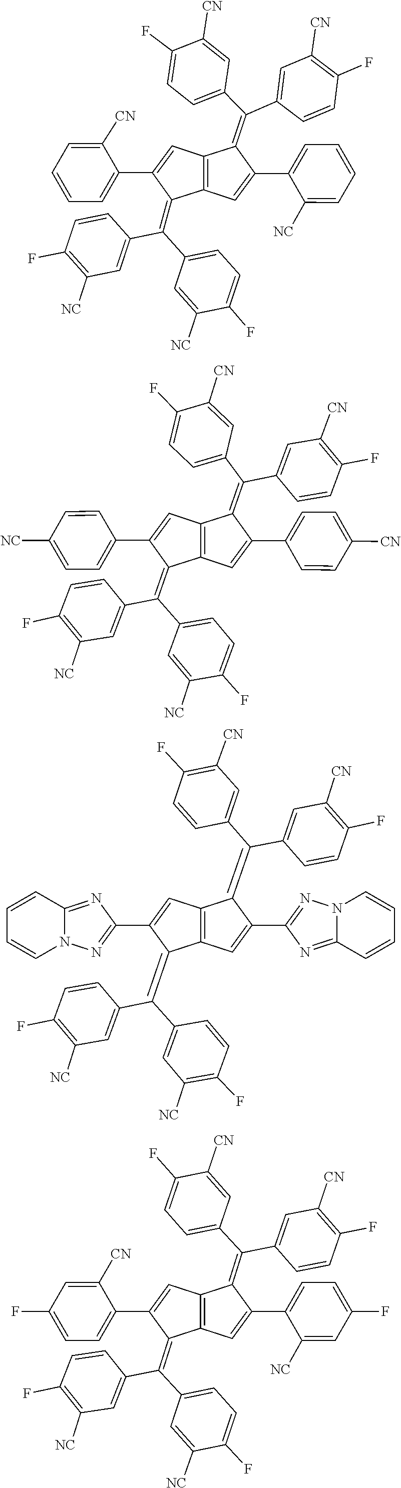

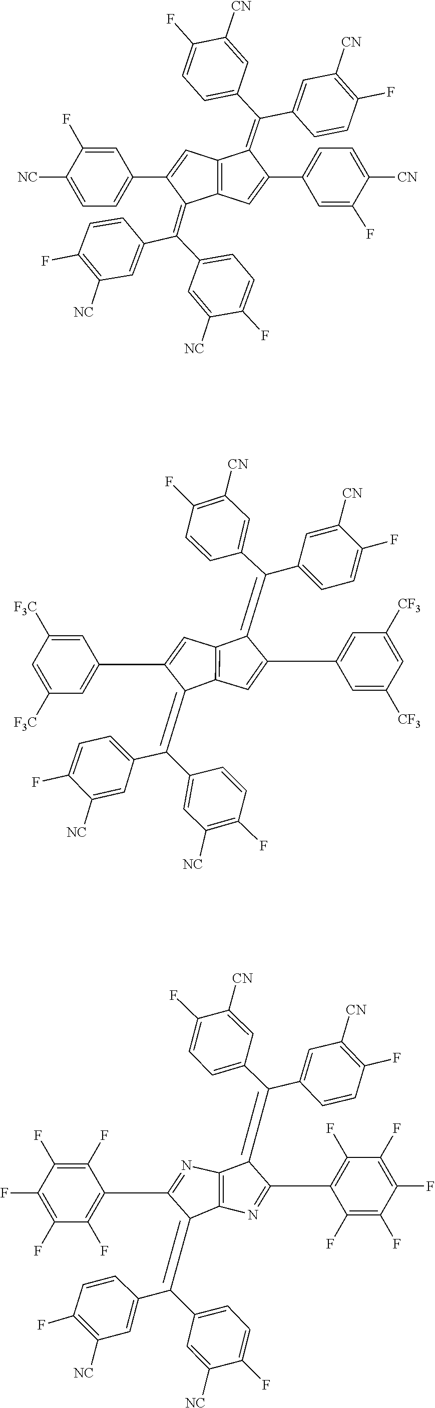 [SMILES: N#Cc1ccc(C2=CC3=C(C=C(c4ccc(C#N)c(F)c4)C3=C(c3ccc(F)c(C#N)c3)c3ccc(F)c(C#N)c3)C2=C(c2ccc(F)c(C#N)c2)c2ccc(F)c(C#N)c2)cc1F ; N#Cc1cc(C(=C2C(c3cc(C(F)(F)F)cc(C(F)(F)F)c3)=CC3=C2C=C(c2cc(C(F)(F)F)cc(C(F)(F)F)c2)C3=C(c2ccc(F)c(C#N)c2)c2ccc(F)c(C#N)c2)c2ccc(F)c(C#N)c2)ccc1F ; N#Cc1cc(C(=C2C(c3c(F)c(F)c(F)c(F)c3F)=NC3=C2N=C(c2c(F)c(F)c(F)c(F)c2F)C3=C(c2ccc(F)c(C#N)c2)c2ccc(F)c(C#N)c2)c2ccc(F)c(C#N)c2)ccc1F]

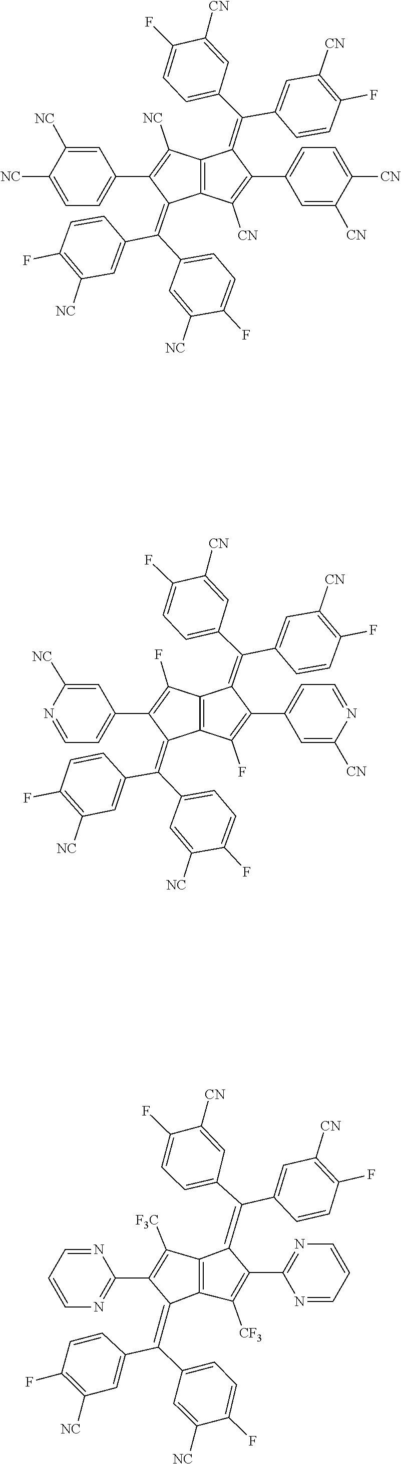 [SMILES: N#CC1=C(c2ccc(C#N)c(C#N)c2)C(=C(c2ccc(F)c(C#N)c2)c2ccc(F)c(C#N)c2)C2=C1C(=C(c1ccc(F)c(C#N)c1)c1ccc(F)c(C#N)c1)C(c1ccc(C#N)c(C#N)c1)=C2C#N ; N#Cc1cc(C2=C(F)C3=C(C(F)=C(c4ccnc(C#N)c4)C3=C(c3ccc(F)c(C#N)c3)c3ccc(F)c(C#N)c3)C2=C(c2ccc(F)c(C#N)c2)c2ccc(F)c(C#N)c2)ccn1 ; N#Cc1cc(C(=C2C3=C(C(=C(c4ccc(F)c(C#N)c4)c4ccc(F)c(C#N)c4)C(c4ncccn4)=C3C(F)(F)F)C(C(F)(F)F)=C2c2ncccn2)c2ccc(F)c(C#N)c2)ccc1F]

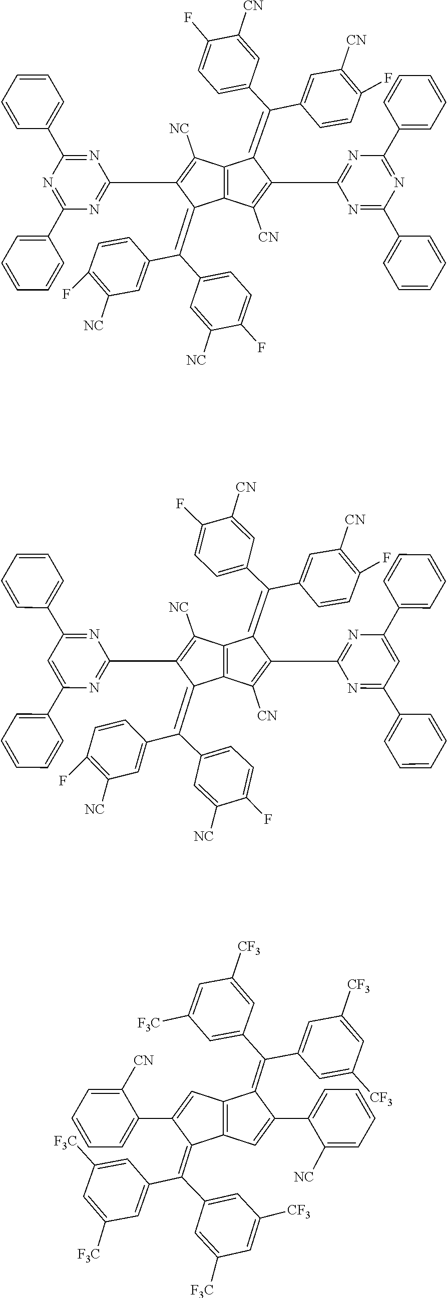 [SMILES: N#CC1=C(c2nc(-c3ccccc3)nc(-c3ccccc3)n2)C(=C(c2ccc(F)c(C#N)c2)c2ccc(F)c(C#N)c2)C2=C1C(=C(c1ccc(F)c(C#N)c1)c1ccc(F)c(C#N)c1)C(c1nc(-c3ccccc3)nc(-c3ccccc3)n1)=C2C#N ; N#CC1=C(c2nc(-c3ccccc3)cc(-c3ccccc3)n2)C(=C(c2ccc(F)c(C#N)c2)c2ccc(F)c(C#N)c2)C2=C1C(=C(c1ccc(F)c(C#N)c1)c1ccc(F)c(C#N)c1)C(c1nc(-c3ccccc3)cc(-c3ccccc3)n1)=C2C#N ; N#Cc1ccccc1C1=CC2=C(C=C(c3ccccc3C#N)C2=C(c2cc(C(F)(F)F)cc(C(F)(F)F)c2)c2cc(C(F)(F)F)cc(C(F)(F)F)c2)C1=C(c1cc(C(F)(F)F)cc(C(F)(F)F)c1)c1cc(C(F)(F)F)cc(C(F)(F)F)c1]

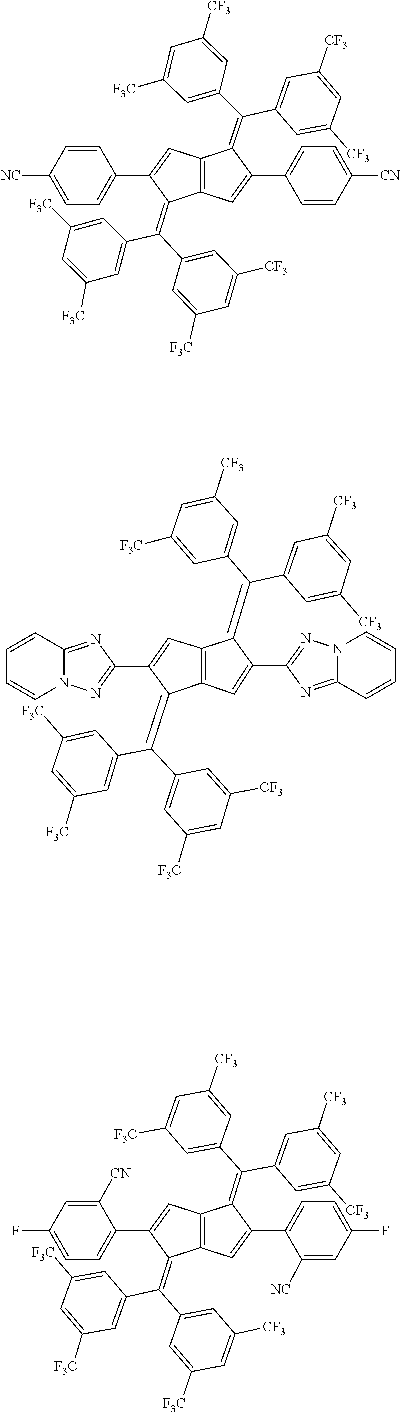

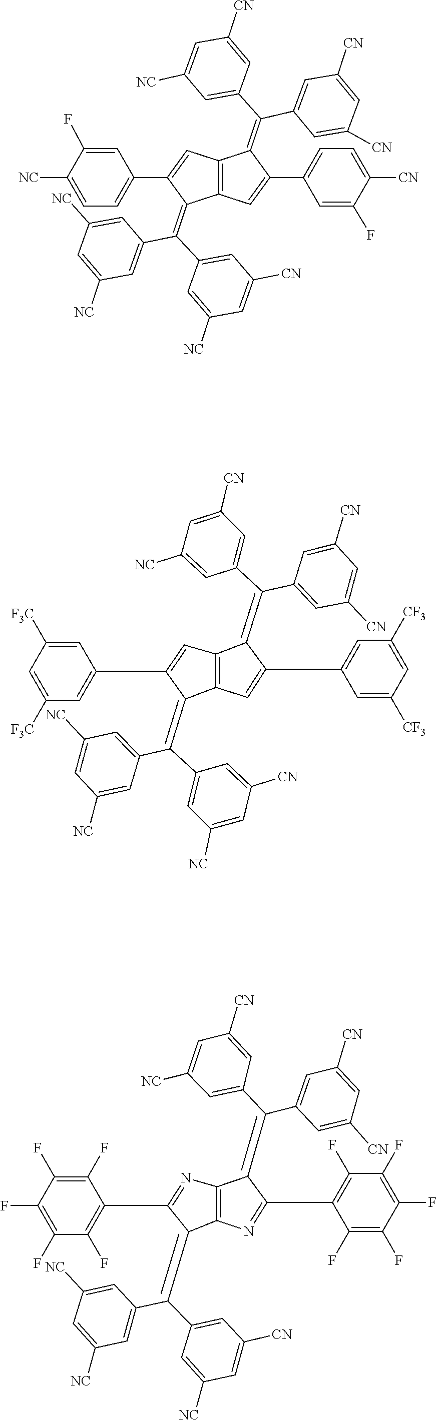 [SMILES: N#Cc1cc(C#N)cc(C(=C2C(c3ccc(C#N)c(F)c3)=CC3=C2C=C(c2ccc(C#N)c(F)c2)C3=C(c2cc(C#N)cc(C#N)c2)c2cc(C#N)cc(C#N)c2)c2cc(C#N)cc(C#N)c2)c1 ; N#Cc1cc(C#N)cc(C(=C2C(c3cc(C(F)(F)F)cc(C(F)(F)F)c3)=CC3=C2C=C(c2cc(C(F)(F)F)cc(C(F)(F)F)c2)C3=C(c2cc(C#N)cc(C#N)c2)c2cc(C#N)cc(C#N)c2)c2cc(C#N)cc(C#N)c2)c1 ; N#Cc1cc(C#N)cc(C(=C2C(c3c(F)c(F)c(F)c(F)c3F)=NC3=C2N=C(c2c(F)c(F)c(F)c(F)c2F)C3=C(c2cc(C#N)cc(C#N)c2)c2cc(C#N)cc(C#N)c2)c2cc(C#N)cc(C#N)c2)c1]

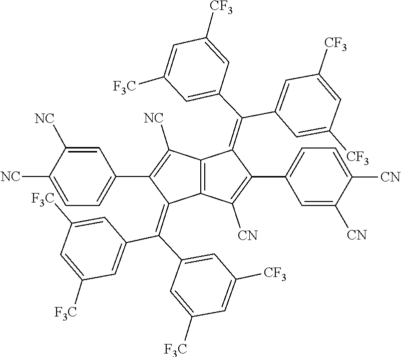 [SMILES: N#CC1=C(c2ccc(C#N)c(C#N)c2)C(=C(c2cc(C(F)(F)F)cc(C(F)(F)F)c2)c2cc(C(F)(F)F)cc(C(F)(F)F)c2)C2=C1C(=C(c1cc(C(F)(F)F)cc(C(F)(F)F)c1)c1cc(C(F)(F)F)cc(C(F)(F)F)c1)C(c1ccc(C#N)c(C#N)c1)=C2C#N]

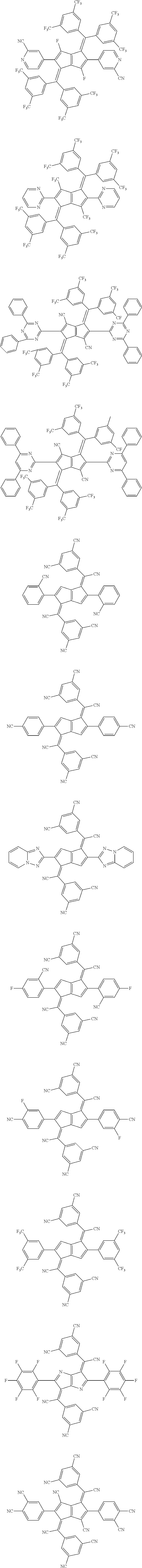

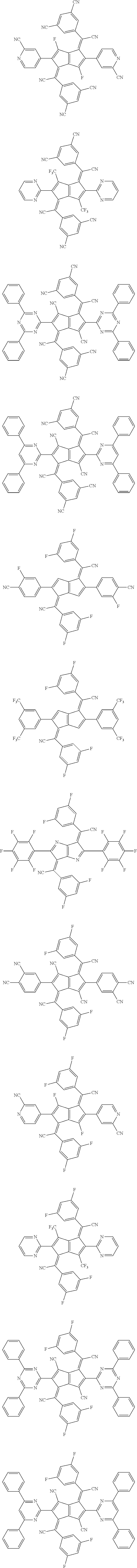

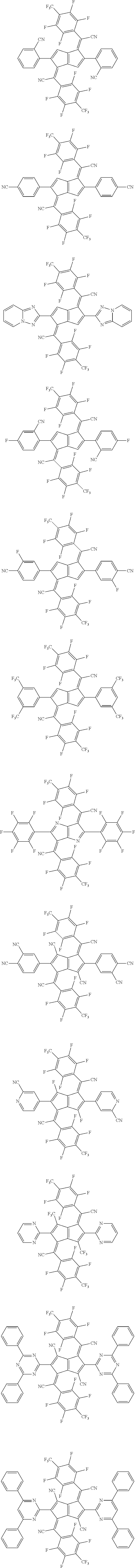

[chemical structure]

[chemical structure]

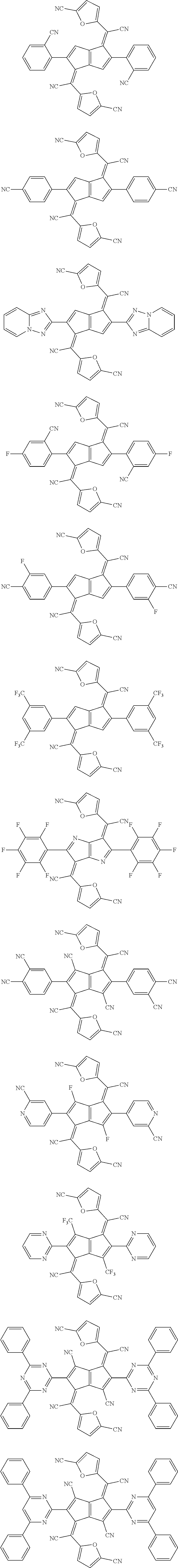

[chemical structure]

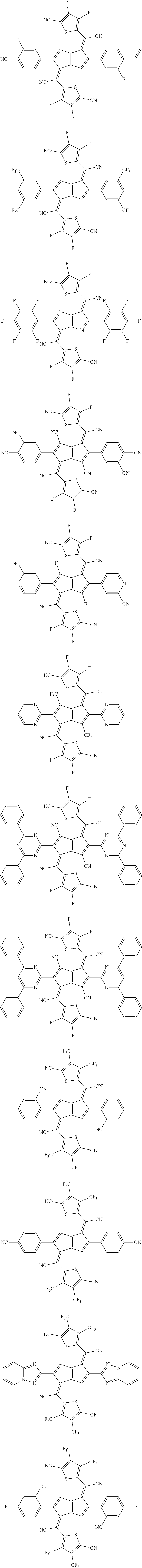

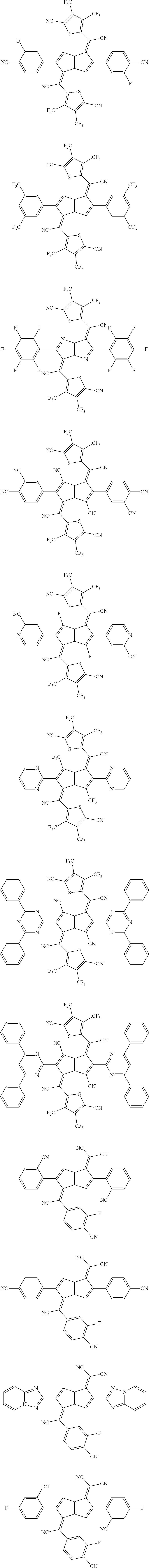

In some embodiments, the LUMO of the organic compound as described herein ≤−4.8 eV, preferably ≤−4.9 eV, more preferably ≤−5.0 eV, further preferably ≤−5.1 eV, and most preferably ≤−5.2 eV.

In some embodiments, ΔLUMO of the organic compound as described herein ≥0.5 eV, preferably ≥0.6 eV, more preferably ≥0.7 eV, and most preferably ≥0.8 eV; where ΔLUMO=(LUMO+1)-LUMO, may be obtained by quantum calculation as described below.

In some embodiments, ΔE_{ST} of the organic compound as described herein ≥0.6 eV, preferably ≥0.7 eV, more preferably ≥0.8 eV, and most preferably ≥0.9 eV; where ΔE_{ST}=S1−T1, S1 stands for the singlet excited energy level, T1 stands for the triplet excited energy level, which can be obtained by quantum calculation as described below.

In some embodiments, the glass transition temperature (Tg) of the organic compound as described herein ≥100° C. In some embodiments, Tg≥110° C. In some embodiments, Tg≥120° C. In some embodiments, Tg≥130° C. In some embodiments, Tg≥140° C.

In some embodiments, the organic compound as described herein is partially deuterated; preferably of 10% or more of total H, more preferably of 20% or more of total H, further preferably of 30% or more of total H, and most preferably of 40% or more of total H, are deuterated.

In some embodiments, the organic compound as described herein is a small molecular material.

In some embodiments, the organic compound as described herein is used for evaporation-based OLEDs. For this purpose, the molecular weight of the organic compound as described herein ≤1200 g/mol, preferably ≤900 g/mol, more preferably ≤850 g/mol, further preferably ≤800 g/mol, and most preferably ≤700 g/mol.

In another aspect, the present disclosure also provides a mixture comprising the organic compound as described herein and at least one organic functional material. The at least one organic functional material is selected from a hole-injection material (HIM), a hole-transport material (HTM), a hole-blocking material (HBM), an electron-injection material (EIM), an electron-transport material (ETM), an electron-blocking material (EBM), an emitting material, a host material (Host), or an organic dye. These organic functional materials are described in detail, for example, in WO2010135519A1, US20090134784A1, and WO2011110277A1. The entire contents of these three documents are hereby incorporated herein for reference.

In some embodiments, the at least one organic functional material of the mixture is selected from a hole-transport material (HTM) or a host material (Host). In some embodiments, the mixture comprises at least one hole-transport material (HTM) and a dopant, where the dopant is an organic compound as described herein, and the molar ratio of the dopant to the hole-transport material ranges from 1:1 to 1:100,000.

The host (triplet host) and HTM are described in detail below (but not limited thereto).

### 1. Triplet Host

Examples of triplet host materials are not specially limited and any metal complex or organic compound may be used as the host material as long as its triplet energy is higher than that of the emitter, especially higher than that of the triplet emitter or a phosphorescent emitter. Examples of metal complexes that may be used as a triplet host include (but not limited to) the following general structures.

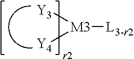

Where M3 is a metal; (Y₃-Y₄) is a bidentate ligand; Y₃ and Y₄ are each independently selected from C, N, O, P, or S; L is an auxiliary ligand; r2 is an integer with the value from 1 to the maximum coordination number of this metal.

In some embodiments, the metal complexes that can be used as the triplet host have the following form:

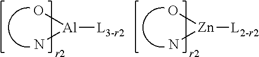

(O—N) is a bidentate ligand in which the metal is coordinated to the O atoms and the N atoms. r2 is an integer with a value from 1 to the maximum coordination number of this metal.

In some embodiments, M3 may be selected from Ir or Pt.

Examples of organic compounds used as triplet hosts are selected from: compounds comprising cyclic aryl groups, such as benzene, biphenyl, triphenyl, benzofluorene; compounds comprising heterocyclic aryl groups, such as dibenzothiophene, dibenzofuran, dibenzoselenophene, furan, thiophene, benzofuran, benzothiophene, benzoselenophene, carbazole, dibenzocarbazole, iodocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, oxazole, dibenzooxazole, benzisoxazole, benzothiazole, quinoline, isoquinoline, o-diazanaphthalene, quinazoline, quinoxaline, naphthalene, phthalein, pteridine, xanthene, acridine, phenazine, phenothiazine, phenoxazine, dibenzoselenophene, benzofuranopyridine, furazopyridine, benzothienopyridine, thienodipyridine, benzoselenophenopyridine, or selenophenodipyridine; and groups comprising 2 to 10 ring structures which may be the same or different types of cyclic aryl or aromatic heterocyclic group and are bonded to each other directly or through at least one of the following groups: oxygen atom, nitrogen atom, sulfur atom, silicon atom, phosphorus atom, boron atom, chain structural unit, or aliphatic cyclic group; and where each Ar may be further optionally substituted, and the substituents may optionally be hydrogen, deuterium, cyano, halogen, alkyl, alkoxy, amino, alkene, alkyne, aralkyl, heteroalkyl, aryl or heteroaryl.

In some embodiments, the triplet host material may be selected from the compounds comprising at least one of the following groups.

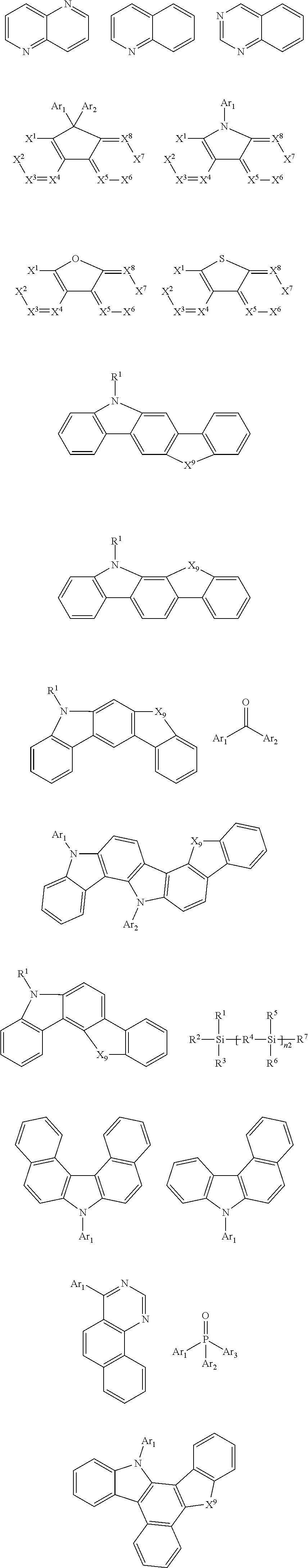

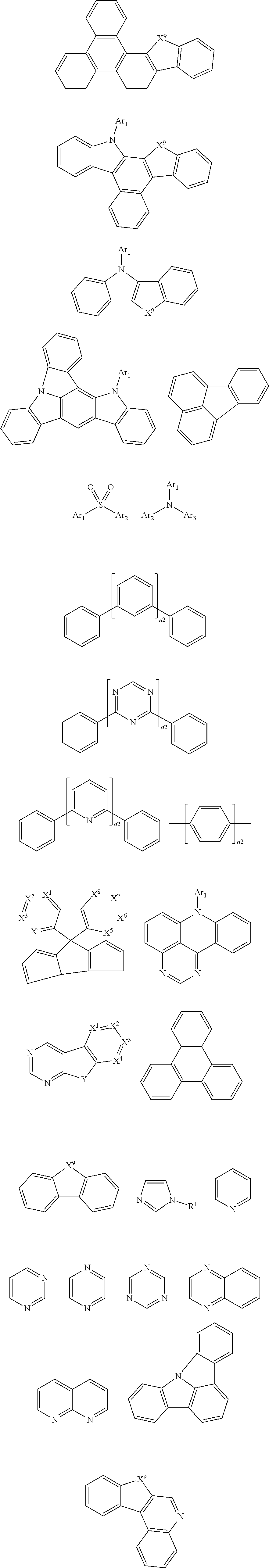

Where R¹ to R⁷ are identically defined as the above-mentioned R₁; each X⁹ is independently selected from C(R)₂, NR, O, or S; each of X¹ to X⁸ is independently selected from CR or N; each of Ar1 to Ar3 is independently selected from an aromatic group or a heteroaromatic group, and each R is independently selected from the group consisting of H, deuterium, halogen atom (F, Cl, Br, I), cyano, alkyl, alkoxy, amino, alkenyl, alkynyl, aralkyl, heteroalkyl, aryl, and heteroaryl; each n2 is an integer from 1 to 20.

Examples of suitable triplet host materials are listed below, but not limited to:

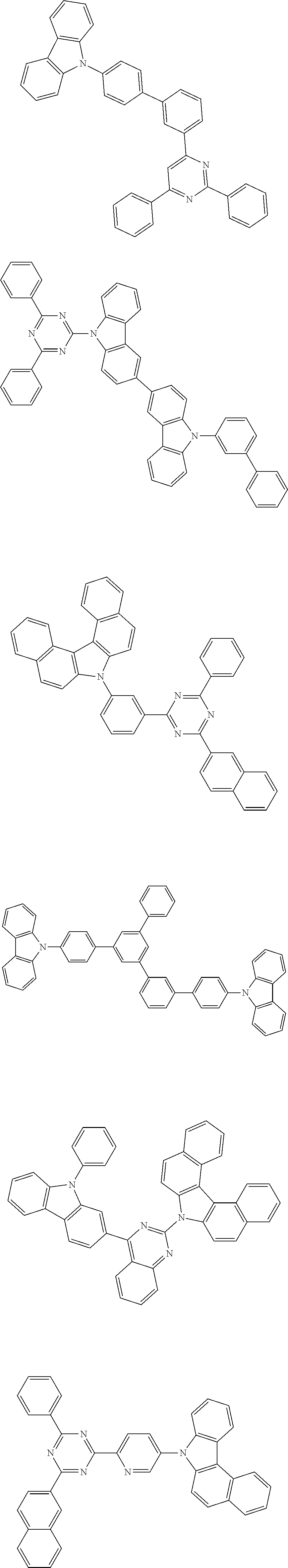

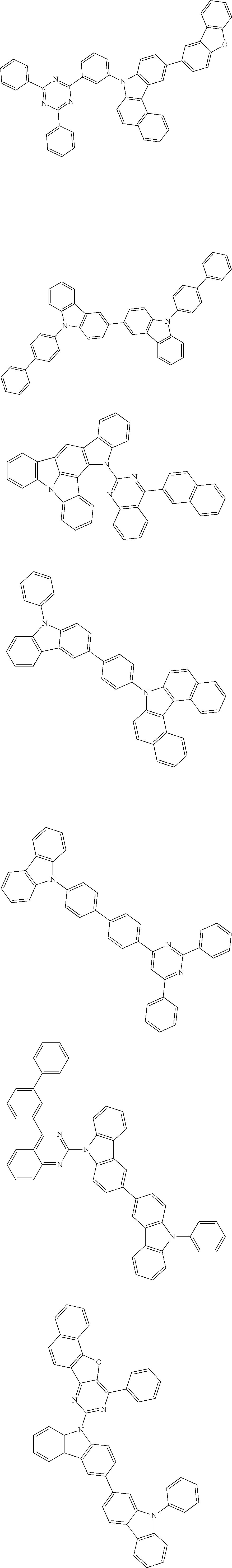

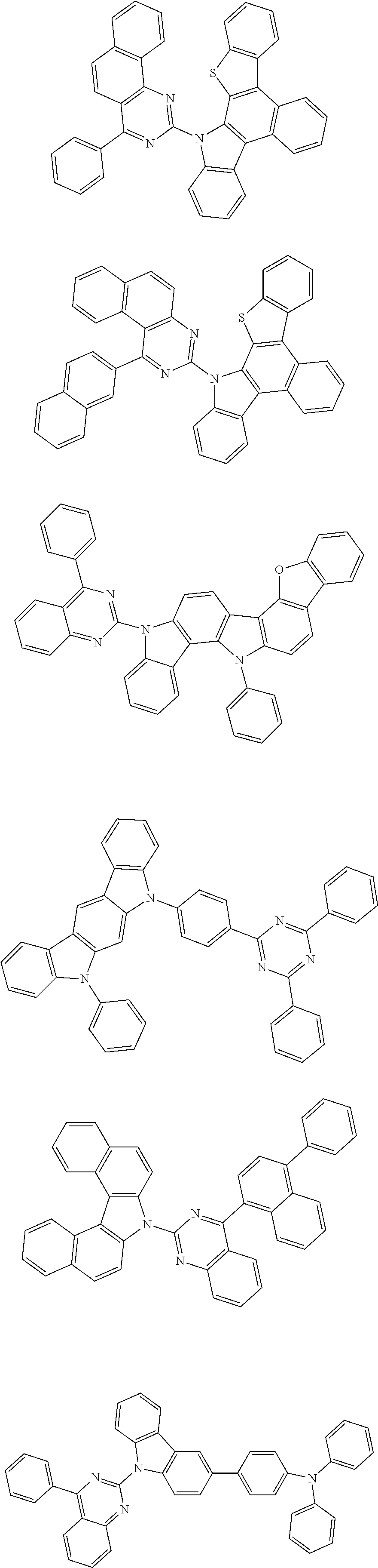 [SMILES: c1ccc(-c2nc(-n3c4ccccc4c4c5ccccc5c5c6ccccc6sc5c43)nc3c2ccc2ccccc23)cc1 ; c1ccc2cc(-c3nc(-n4c5ccccc5c5c6ccccc6c6c7ccccc7sc6c54)nc4ccc5ccccc5c34)ccc2c1 ; c1ccc(-c2nc(-n3c4ccccc4c4c3ccc3c5c6oc7ccccc7c6ccc5n(-c5ccccc5)c34)nc3ccccc23)cc1 ; c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2ccc(-c3nc(-n4c5ccc6ccccc6c5c5c6ccccc6ccc54)nc4ccccc34)c3ccccc23)cc1 ; c1ccc(-c2nc(-n3c4ccccc4c4cc(-c5ccc(N(c6ccccc6)c6ccccc6)cc5)ccc43)nc3ccccc23)cc1]

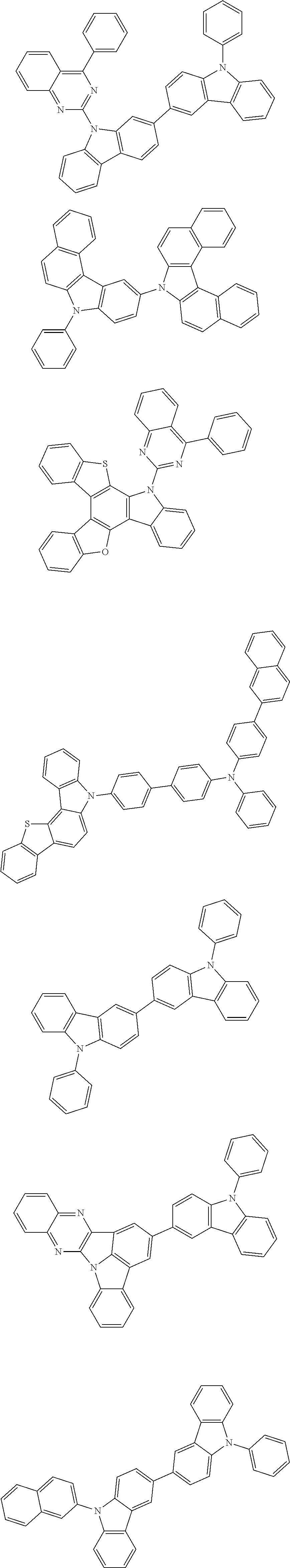

### 2. HTM

Suitable organic HTM materials may include any one or any combination of the compounds having the following structural units: phthalocyanines, porphyrins, amines, aryl amines, biphenyl triaryl amines, thiophenes, bithiophenes, pyrroles, anilines, carbazoles, indenofluorenes, or derivatives thereof.

Examples of cyclic aromatic amine-derived compounds that can be used as HTM include, but not limited to the general structures as follows:

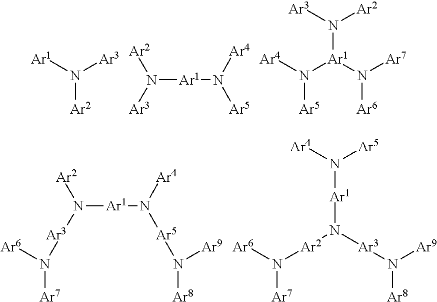

Each of Ar¹ to Ar⁹ may be independently selected from: cyclic aryl groups such as benzene, biphenyl, triphenyl, benzo, naphthalene, anthracene, phenalene, phenanthrene, fluorene, pyrene, chrysene, perylene, azulene; and aromatic heterocyclic groups such as dibenzothiophene, dibenzofuran, furan, thiophene, benzofuran, benzothiophene, benzoselenophene, carbazole, pyrazole, imidazole, triazole, isoxazole, thiazole, oxadiazole, oxatriazole, dioxazole, thiadiazole, pyridine, pyridazine, pyrimidine, pyrazine, triazine, oxazine, oxathiazin, oxadiazine, indole, benzimidazole, indazole, indoxazine, bisbenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, dibenzoselenophene, benzoselenophene, benzofuropyridine, indolocarbazole, pyridylindole, pyrrolodipyridine, furodipyridine, benzothienopyridine, thienodipyridine, benzoselenophenopyridine, or selenophenodipyridine; groups comprising 2 to 10 ring structures which may be the same or different types of cyclic aryl or aromatic heterocyclic group and are bonded to each other directly or through at least one of the following groups, such as: oxygen atom, nitrogen atom, sulfur atom, silicon atom, phosphorus atom, boron atom, chain structural unit, or aliphatic cyclic group; and wherein each of Ar¹ to Ar⁹ may be further arbitrarily substituted, and the substituents may optionally be H, D, alkyl, alkoxy, amino, alkene, alkyne, aralkyl, heteroalkyl, aryl, or heteroaryl.

In some embodiments, each of Ar¹ to Ar⁹ may be independently selected from the group consisting of:

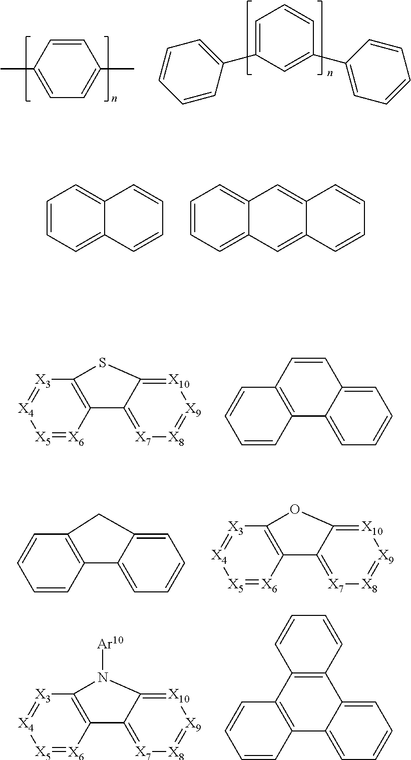

Where n is an integer from 1 to 20; each of X₃ to X₁₀ is CH or N; Ar¹⁰ is defined as the above-mentioned Ar¹.

Additional examples of cyclic aryl amine-derived compounds may be found in U.S. Pat. Nos. 3,567,450, 4,720,432, 5,061,569, and 3,615,404.

Examples of metal complexes that may be used as HTM include, but not limited to the general structures as follows: M-L3-in

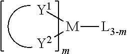

M is a metal having an atomic weight greater than 40. In some embodiments, M is selected from Ir, Pt, Os, or Zn.

(Y¹-Y²) is a bidentate ligand, where each of Y¹ and Y² is independently selected from C, N, O, P, or S; L is an auxiliary ligand; m is an integer from 1 to the maximum coordination number of the metal.

In some embodiments, (Y¹-Y²) is a 2-phenylpyridine derivative. In some embodiments, (Y¹-Y²) is a carbene ligand. In another aspect, the HOMO of the metal complex is greater than −5.5 eV (relative to the vacuum level).

Suitable examples of HTM compounds are listed below:

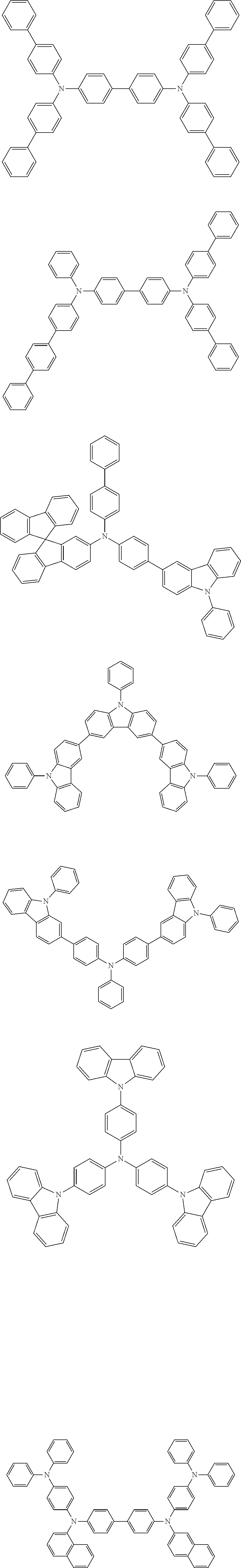

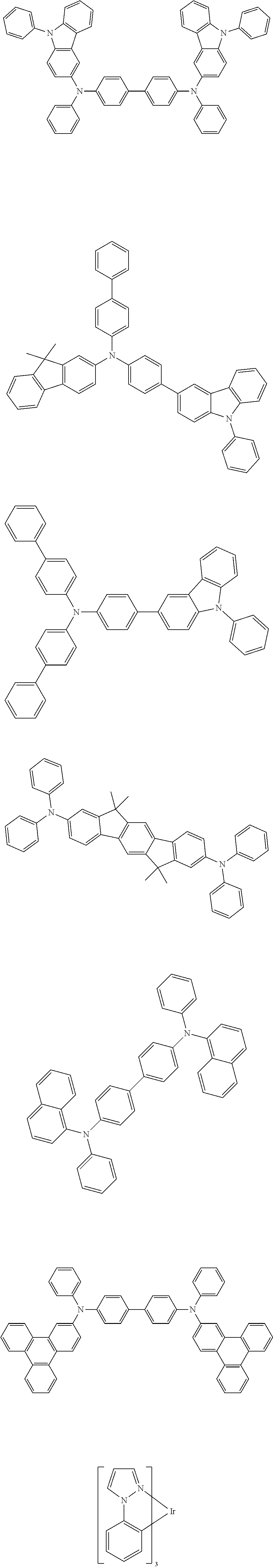

In yet another aspect, the present disclosure further provides a formulation or an ink comprising an organic compound or a mixture as described herein, and at least one organic solvent.

The viscosity and surface tension of the ink are important parameters in printing processes. A suitable ink surface tension is required for the specific substrates and the specific printing methods.

In some embodiments, the surface tension of the ink as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink as described herein at 25° C. is in the range of from about 1 cps to 100 cps; particularly in the range of 1 cps to 50 cps; more particularly in the range of 1.5 cps to 20 cps; and most particularly in the range of 4.0 cps to 20 cps. The resulting formulation will be particularly suitable for ink-jet printing.

The viscosity can be adjusted by different methods, such as by the selection of appropriate organic solvent and the concentration of the functional materials in the ink. In the ink comprising the above-mentioned organic compounds or polymers as described herein facilitate the adjustment of the printing ink in the appropriate range according to the printing method used. Generally, in the formulation comprising the functional material as described herein, the weight ratio of the functional material ranges from 0.3 wt % to 30 wt %, preferably in the range of 0.5 wt % to 20 wt %, more preferably in the range of 0.5 wt % to 15 wt %, further preferably in the range of 0.5 wt % to 10 wt %, and most preferably in the range of 1 wt % to 5 wt %.

In the formulation as described herein, the at least one organic solvent is selected from aromatics, heteroaromatics, esters, aromatic ketones, aromatic ethers, aliphatic ketones, aliphatic ethers, alicyclic or olefinic compounds, borate, phosphorate, or mixtures of two or more of them.

In some embodiments, in the formulation as described herein, the at least one organic solvent is selected from aromatic or heteroaromatic-based solvents.

Examples of aromatic or heteroaromatic-based solvents suitable for the present disclosure include, but not limited to: p-diisopropylbenzene, amylbenzene, tetrahydronaphthalene, cyclohexylbenzene, chloronaphthalene, 1,4-dimethylnaphthalene, 3-isopropylbenzene, p-methylisopropylbenzene, dipentylbenzene, tripentylbenzene, pentyltoluene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, cyclohexylbenzene, benzylbutylbenzene, dimethylnaphthalene, 3-isopropylbiphenyl, p-methylisopropylbenzene, 1-methylnaphthalene, 1,2,4-trichlorobenzene, 4,4-difluorobenzenemethane, 1,2-dimethoxy-4-(1-propenyl)benzene, diphenylmethane, 2-phenylpyridine, 3-phenylpyridine, N-methyldiphenylamine, 4-isopropylbiphenyl, dichlorodiphenylmethane, 4-(3-phenylpropyl)pyridine, benzyl benzoate, 1,1-bis(3,4-dimethylphenyl)ethane, 2-isopropylnaphthalene, quinoline, isoquinoline, methyl 2-furancarboxylate, ethyl 2-furanicarboxylate, etc.

Examples of aromatic ketone-based solvents suitable for the present disclosure include, but not limited to: 1-tetrahydronaphthalene, 2-tetrahydronaphthalene, 2-(phenylepoxy)tetrahydronaphthalene, 6-(methoxy)tetrahydronaphthalene, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, etc.

Examples of aromatic ether-based solvents suitable for the present disclosure include, but not limited to: 3-phenoxytoluene, butoxybenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,3-dipropoxybenzene, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether.

In some embodiments, in the formulation of the present disclosure, the at least one organic solvent can be selected from aliphatic ketones, such as, 2-nonanone, 3-nonanone, 5-nonanone, 2-decanone, 2,5-hexanedione, 2,6,8-trimethyl-4-nonanone, fenchone, phoron, isophorone, di-n-amyl ketone, etc; and the at least one organic solvent of the present disclosure can be selected from aliphatic ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, in the formulation of the present disclosure, the at least one organic solvent can be selected from: ester-based solvents including alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc. Particular preferred as octyl octanoate, diethyl sebacate, diallyl phthalate and isononyl isononanoate.

The solvent may be used alone or as mixtures of two or more organic solvents.

In some embodiments, the formulation of the present disclosure comprises an organic compound or a mixture as described herein, and at least one organic solvent, and can further comprise another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or mixtures thereof.

In yet another aspect, the present disclosure further provides the use of the formulation as coatings or printing inks in the preparation of organic electronic devices, particularly preferably by printing or coating processing methods.

Where suitable printing or coating techniques include, but are not limited to, gravure printing, ink-jet printing, typographic printing, screen printing, dip coating, spin coating, blade coating, roller printing, torsion roll printing, planographic printing, flexographic printing, rotary printing, spray printing, brush coating, pad printing, slit die coating, and so on. Preferred techniques are gravure printing, screen printing, and ink-jet printing. Gravure printing and ink-jet printing will be applied in the embodiments of the present disclosure. The solution or dispersion may additionally comprise one or more components, such as surfactants, lubricants, wetting agents, dispersing agents, hydrophobic agents, binders, etc, which are used to adjust the viscosity and film forming properties, or to improve adhesion, etc. For more information about printing technologies and their requirements for solutions, such as solvent, concentration, viscosity, etc, please refer to “Handbook of Print Media: Technologies and Production Methods”, edited by Helmut Kipphan, ISBN 3-540-67326-1.

The preparation methods as described above, where the formed functional layer has a thickness of 5 nm-1000 nm.

In yet another aspect, the present disclosure further provides the use of the organic compound or mixture as described herein in organic electronic devices.

In yet another aspect, the present disclosure further provides an organic electronic device comprising an organic compound or a mixture as described herein, or is prepared using a formulation as described herein. In some embodiments, the organic electronic device comprises a functional layer, the functional layer comprises an organic compound or a mixture as described herein, or is prepared using a formulation as described herein.

The organic electronic device may be selected from, but not limited to an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, or an organic plasmon emitting diode (OPED), etc, particularly preferably an organic electroluminescent device and a photovoltaic cell, such as an OLED, an OPV, an OLEEC, an organic light emitting field effect transistor.

In some embodiments, the functional layer is selected from a hole-injection layer or a hole-transport material.

In some embodiments, the organic electronic device comprises a hole-injection layer or a hole-transport layer, where the hole-injection/hole-transport layer comprises an organic compound or a mixture as described herein.

In the organic electroluminescent devices as described herein, in particular an OLED, which comprises a substrate, an anode, at least one light-emitting layer, and a cathode.

The substrate should be opaque or transparent. A transparent substrate could be used to produce a transparent light-emitting device (for example: Bulovic et al., Nature 1996, 380, p 29, and Gu et al., Appl. Phys. Lett. 1996, 68, p 2606). Substrate may be either rigid or elastic. The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature (Tg) over 150° C., preferably over 200° C., more preferably over 250° C., and most preferably over 300° C. Examples of the suitable flexible substrate includes polyethylene terephthalate (PET) and polyethylene glycol (2,6-naphthalene) (PEN).

The choice of anodes may include a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level of the emitter of the light-emitting layer, or the HOMO energy level/valence band energy level of the p-type semiconductor materials of the hole-injection layer (HIL)/hole-transport layer (HTL)/electron-blocking layer (EBL)<0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal or a metal oxide. The cathode should be able to easily inject electrons into the EIL, the ETL, or the directly into the light-emitting layer. In some embodiments, the absolute value of the difference between the work function of the cathode and the LUMO energy level of the emitter of the light-emitting layer, or the LUMO energy level/conduction band energy level of the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials that can be used as cathodes for OLEDs may be applied as cathode materials for the devices as described herein. Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode materials can be deposited using any suitable technique, such as the suitable physical vapor deposition method, including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1, the entire contents of these three documents are hereby incorporated herein for reference.

The light-emitting device as described herein has a light-emitting wavelength between 300 nm and 1000 nm, preferably between 350 nm and 900 nm, more preferably between 400 nm and 800 nm.

In yet another aspect, the present disclosure further provides the applications of organic electronic devices in various electronic equipment, including, but not limited to, display devices, lighting equipment, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

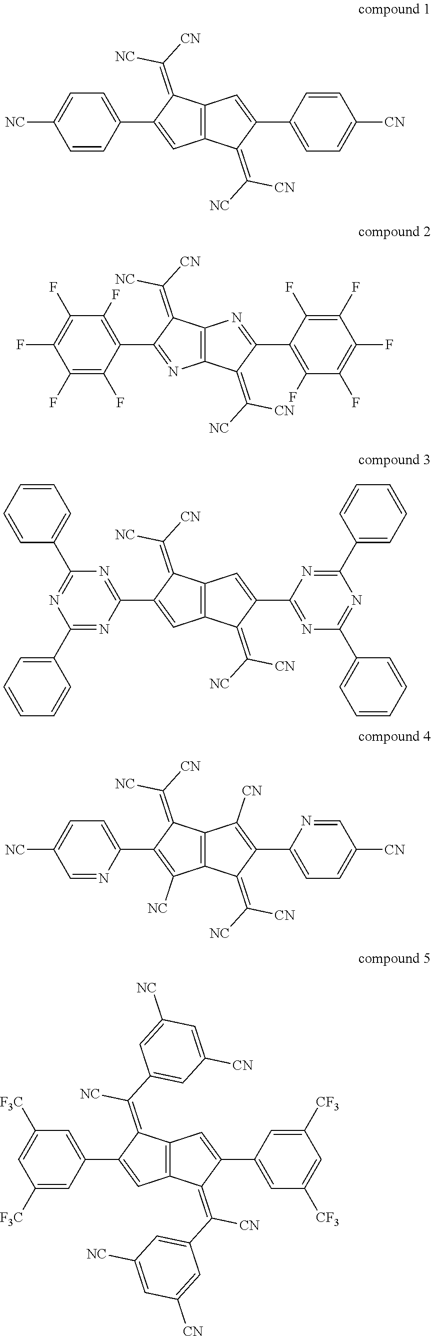 [SMILES: N#CC(C#N)=C1C(c2ccc(C#N)cc2)=CC2=C1C=C(c1ccc(C#N)cc1)C2=C(C#N)C#N ; N#CC(C#N)=C1C(c2c(F)c(F)c(F)c(F)c2F)=NC2=C1N=C(c1c(F)c(F)c(F)c(F)c1F)C2=C(C#N)C#N ; N#CC(C#N)=C1C(c2nc(-c3ccccc3)nc(-c3ccccc3)n2)=CC2=C1C=C(c1nc(-c3ccccc3)nc(-c3ccccc3)n1)C2=C(C#N)C#N ; N#CC(C#N)=C1C2=C(C(C#N)=C1c1ccc(C#N)cn1)C(=C(C#N)C#N)C(c1ccc(C#N)cn1)=C2C#N ; N#C/C(=C1\C(c2cc(C(F)(F)F)cc(C(F)(F)F)c2)=CC2=C1C=C(c1cc(C(F)(F)F)cc(C(F)(F)F)c1)/C2=C(\C#N)c1cc(C#N)cc(C#N)c1)c1cc(C#N)cc(C#N)c1]

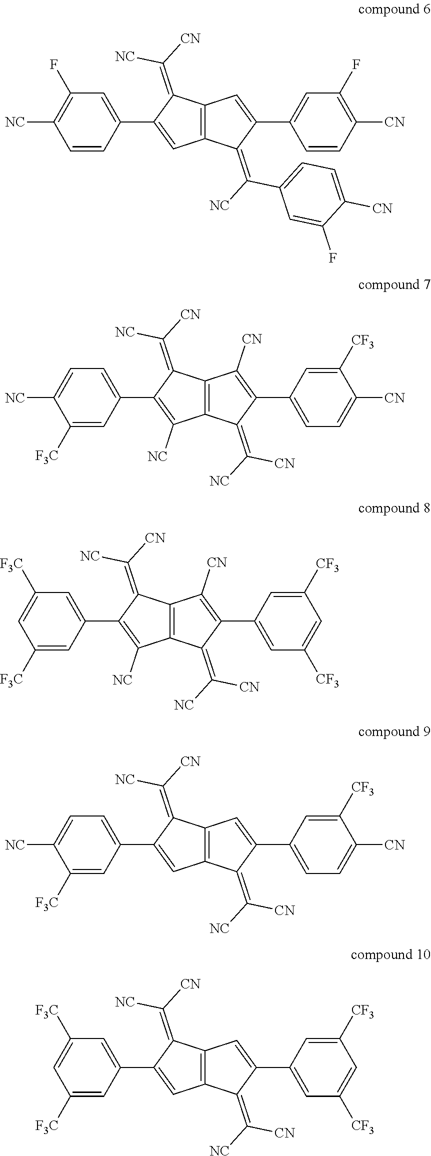 [SMILES: N#CC(C#N)=C1C(c2ccc(C#N)c(F)c2)=CC2=C1C=C(c1ccc(C#N)c(F)c1)/C2=C(/C#N)c1ccc(C#N)c(F)c1 ; N#CC(C#N)=C1C2=C(C(C#N)=C1c1ccc(C#N)c(C(F)(F)F)c1)C(=C(C#N)C#N)C(c1ccc(C#N)c(C(F)(F)F)c1)=C2C#N ; N#CC(C#N)=C1C2=C(C(C#N)=C1c1cc(C(F)(F)F)cc(C(F)(F)F)c1)C(=C(C#N)C#N)C(c1cc(C(F)(F)F)cc(C(F)(F)F)c1)=C2C#N ; N#CC(C#N)=C1C(c2ccc(C#N)c(C(F)(F)F)c2)=CC2=C1C=C(c1ccc(C#N)c(C(F)(F)F)c1)C2=C(C#N)C#N ; N#CC(C#N)=C1C(c2cc(C(F)(F)F)cc(C(F)(F)F)c2)=CC2=C1C=C(c1cc(C(F)(F)F)cc(C(F)(F)F)c1)C2=C(C#N)C#N]

### Synthetic Example 1

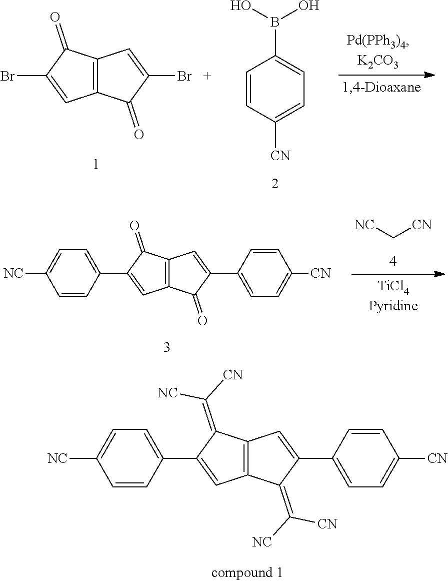 [SMILES: O=c1c(Br)cc2c(=O)c(Br)cc1=2 ; N#Cc1ccc(B(O)O)cc1 ; N#Cc1ccc(-c2cc3c(=O)c(-c4ccc(C#N)cc4)cc=3c2=O)cc1 ; N#CCC#N ; N#CC(C#N)=C1C(c2ccc(C#N)cc2)=CC2=C1C=C(c1ccc(C#N)cc1)C2=C(C#N)C#N]

### Synthesis of Intermediate 3

Intermediate 1 (4.97 g, 17.25 mmol) and intermediate 2 (5.58 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 4.90 g (84.5% yield) of compound 1.

### Synthesis of Compound 1

Intermediate 3 (2.00 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 1 (0.77 g, 1.78 mmol).

### Synthetic Example 2

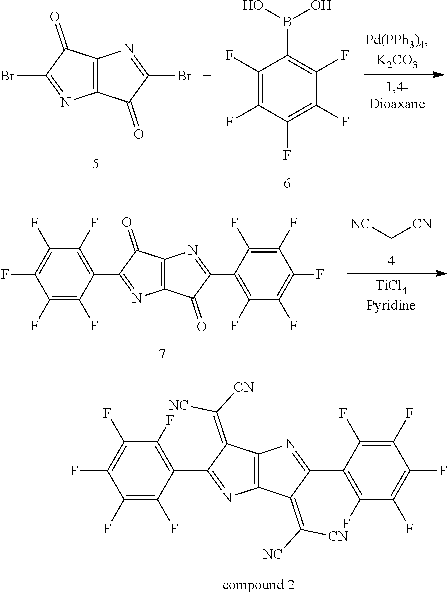 [SMILES: O=c1c(Br)nc2c(=O)c(Br)nc1=2 ; OB(O)c1c(F)c(F)c(F)c(F)c1F ; O=c1c(-c2c(F)c(F)c(F)c(F)c2F)nc2c(=O)c(-c3c(F)c(F)c(F)c(F)c3F)nc1=2 ; N#CCC#N ; N#CC(C#N)=C1C(c2c(F)c(F)c(F)c(F)c2F)=NC2=C1N=C(c1c(F)c(F)c(F)c(F)c1F)C2=C(C#N)C#N]

### Synthesis of Intermediate 7

Intermediate 5 (4.97 g, 17.26 mmol) and intermediate 6 (8.05 g, 37.98 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 6.54 g (81.3% yield) of intermediate 7.

### Synthesis of Compound 2

Intermediate 7 (2.00 g, 4.29 mmol) and intermediate 4 (0.85 g, 12.87 mmol) were added to a 250 mL round bottom flask, then the mixture was pour into dichloromethane (250 mL). After cooling down to 0° C., titanium tetrachloride (1.41 mL, 12.87 mol) and pyridine (1.91 mL, 25.74 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 2 (0.69 g, 1.23 mmol).

### Synthetic Example 3

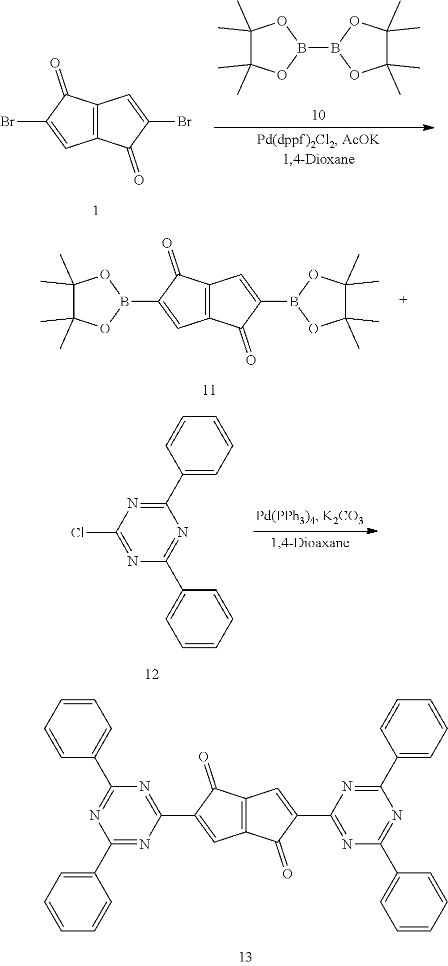

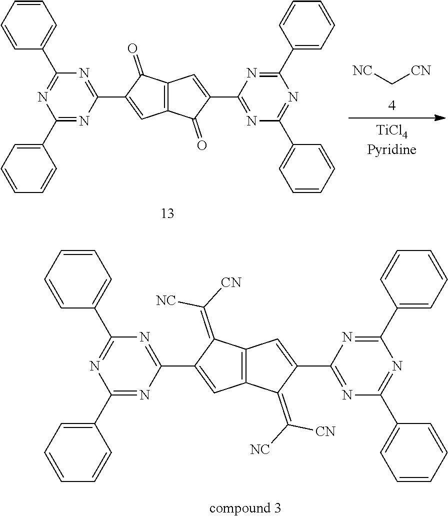 [SMILES: O=c1c(-c2nc(-c3ccccc3)nc(-c3ccccc3)n2)cc2c(=O)c(-c3nc(-c4ccccc4)nc(-c4ccccc4)n3)cc1=2 ; N#CCC#N ; N#CC(C#N)=C1C(c2nc(-c3ccccc3)nc(-c3ccccc3)n2)=CC2=C1C=C(c1nc(-c3ccccc3)nc(-c3ccccc3)n1)C2=C(C#N)C#N]

### Synthesis of Intermediate 11

Compound 1 (4.97 g, 17.25 mmol), bis(pinacolato)diboron (13.14 g, 51.75 mmol), potassium acetate (8.46 g, 86.25 mmol), and 1,4 dioxane (100 ml) were added to a 250 mL three-necked flask. After bubbling for 30 minutes, catalyst Pd(dppf)₂Cl₂ (0.30 g) was added to the above mixture in argon atmosphere, then heated to 90° C. and refluxed for 24 h. After the reaction was completed and cooled down to room temperature, dichloromethane was added into the system to dissolve, then the solvent was removed. After washing and extracting with dichloromethane and water for several times, the reaction solution was removed to obtain crude product, then the resulting sample was further purified by silica gel column chromatography to yield 4.28 g (about 64.3% yield) of intermediate 11.

### Synthesis of Intermediate 13

Intermediate 11 (4.0 g, 10.35 mmol), intermediate 12 (6.08 g, 22.77 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 5.29 g (85.7% yield) of intermediate 13.

### Synthesis of Compound 3

Intermediate 13 (2.56 g, 4.29 mmol) and intermediate 4 (0.85 g, 12.87 mmol) were added to a 250 mL round bottom flask, then the mixture was pour into dichloromethane (250 mL). After cooling down to 0° C., titanium tetrachloride (1.41 mL, 12.87 mol) and pyridine (1.91 mL, 25.74 mol) were added slowly and successively dropwise into the system, then resulting mixture was stirred at room temperature for 24 h. After the reaction was completed, the obtained crude product was dissolved in dichloromethane and the resulting organic phase was washed with water for three times. After the separation and concentration, the result was extracted with dichloromethane, ethyl acetate, and n-hexane. After that, dichloromethane and petroleum ether were added to the result to form a precipitated liquid, then filtered to obtain compound 3 (0.77 g, 1.12 mmol).

### Synthetic Example 4

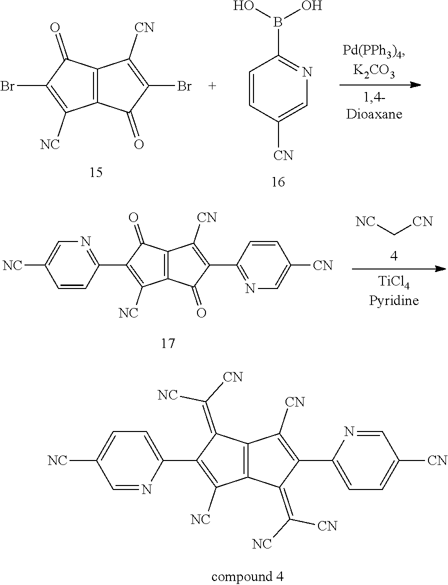 [SMILES: N#Cc1c(Br)c(=O)c2c(C#N)c(Br)c(=O)c1=2 ; N#Cc1ccc(B(O)O)nc1 ; N#Cc1ccc(-c2c(C#N)c3c(=O)c(-c4ccc(C#N)cn4)c(C#N)c=3c2=O)nc1 ; N#CCC#N ; N#CC(C#N)=C1C2=C(C(C#N)=C1c1ccc(C#N)cn1)C(=C(C#N)C#N)C(c1ccc(C#N)cn1)=C2C#N]

### Synthesis of Intermediate 17

Intermediate 15 (5.86 g, 17.25 mmol) and intermediate 16 (5.62 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 5.10 g (76.6% yield) of intermediate 17.

### Synthesis of Compound 4

Intermediate 17 (2.30 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 4 (0.60 g, 1.25 mmol).

### Synthetic Example 5

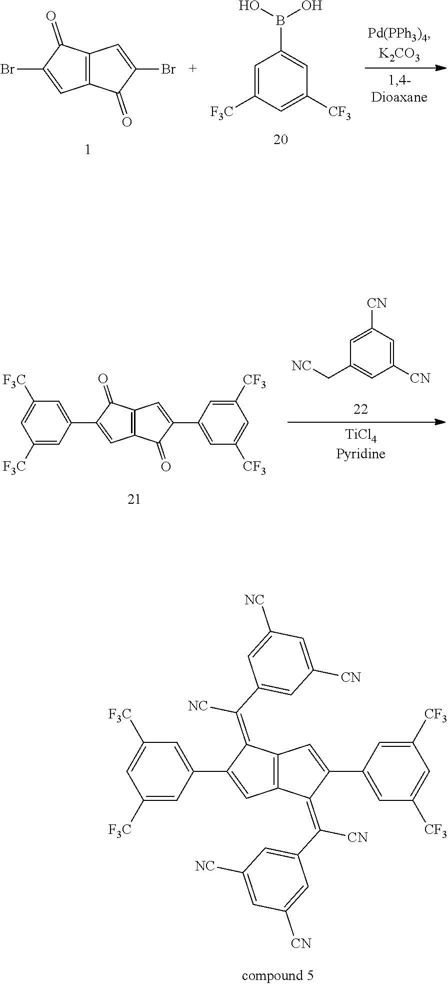 [SMILES: O=c1c(Br)cc2c(=O)c(Br)cc1=2 ; OB(O)c1cc(C(F)(F)F)cc(C(F)(F)F)c1 ; N#CCc1cc(C#N)cc(C#N)c1 ; O=c1c(-c2cc(C(F)(F)F)cc(C(F)(F)F)c2)cc2c(=O)c(-c3cc(C(F)(F)F)cc(C(F)(F)F)c3)cc1=2 ; N#C/C(=C1\C(c2cc(C(F)(F)F)cc(C(F)(F)F)c2)=CC2=C1C=C(c1cc(C(F)(F)F)cc(C(F)(F)F)c1)/C2=C(\C#N)c1cc(C#N)cc(C#N)c1)c1cc(C#N)cc(C#N)c1]

### Synthesis of Intermediate 21

Intermediate 1 (4.97 g, 17.25 mmol) and intermediate 20 (9.79 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 7.27 g (75.6% yield) of intermediate 21.

### Synthesis of Compound 5

Intermediate 21 (3.32 g, 5.95 mmol) and intermediate 22 (2.99 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 5 (0.91 g, 1.06 mmol).

### Synthetic Example 6

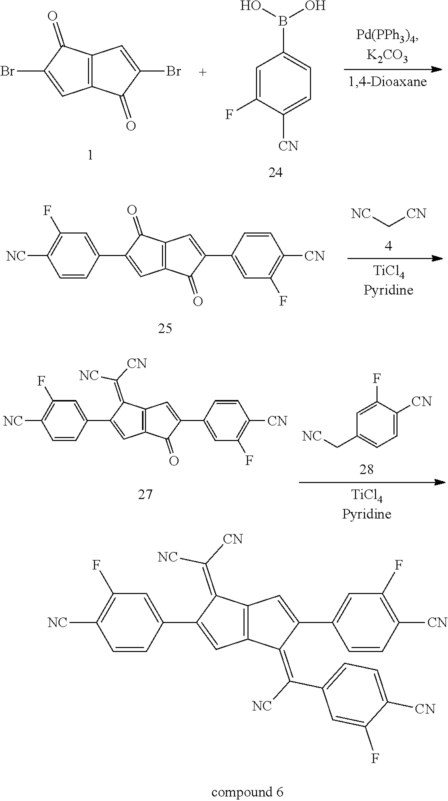

### Synthesis of Intermediate 25

Intermediate 1 (4.97 g, 17.25 mmol) and intermediate 24 (6.26 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 4.96 g (77.3% yield) of intermediate 25.

### Synthesis of Intermediate 27

Intermediate 25 (2.21 g, 5.95 mmol) and intermediate 4 (0.59 g, 8.93 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain intermediate 27 (0.56 g, 1.33 mmol).

### Synthesis of Compound 6

Intermediate 27 (2.21 g, 5.95 mmol) and intermediate 28 (1.43 g, 8.93 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 6 (0.87 g, 1.56 mmol).

### Synthetic Example 7

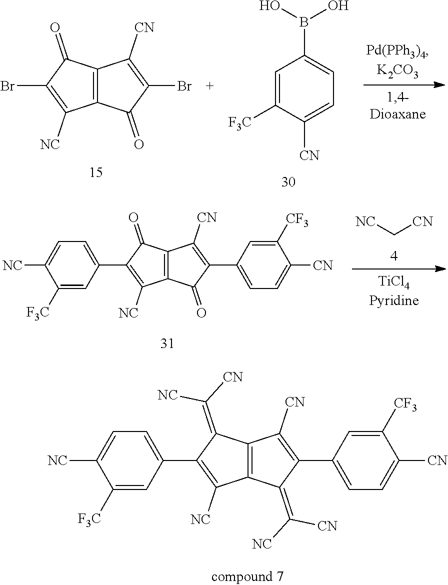 [SMILES: N#Cc1c(Br)c(=O)c2c(C#N)c(Br)c(=O)c1=2 ; N#Cc1ccc(B(O)O)cc1C(F)(F)F ; N#Cc1ccc(-c2c(C#N)c3c(=O)c(-c4ccc(C#N)c(C(F)(F)F)c4)c(C#N)c=3c2=O)cc1C(F)(F)F ; N#CCC#N ; N#CC(C#N)=C1C2=C(C(C#N)=C1c1ccc(C#N)c(C(F)(F)F)c1)C(=C(C#N)C#N)C(c1ccc(C#N)c(C(F)(F)F)c1)=C2C#N]

### Synthesis of Intermediate 31

Intermediate 15 (5.86 g, 17.25 mmol) and intermediate 30 (8.16 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 6.52 g (72.6% yield) of intermediate 31.

### Synthesis of Compound 7

Intermediate 31 (3.10 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 7 (0.71 g, 1.15 mmol).

### Synthetic Example 8

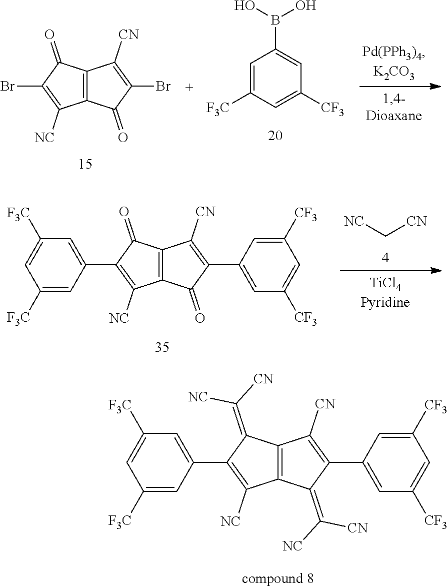 [SMILES: N#Cc1c(Br)c(=O)c2c(C#N)c(Br)c(=O)c1=2 ; OB(O)c1cc(C(F)(F)F)cc(C(F)(F)F)c1 ; N#Cc1c(-c2cc(C(F)(F)F)cc(C(F)(F)F)c2)c(=O)c2c(C#N)c(-c3cc(C(F)(F)F)cc(C(F)(F)F)c3)c(=O)c1=2 ; N#CCC#N ; N#CC(C#N)=C1C2=C(C(C#N)=C1c1cc(C(F)(F)F)cc(C(F)(F)F)c1)C(=C(C#N)C#N)C(c1cc(C(F)(F)F)cc(C(F)(F)F)c1)=C2C#N]

### Synthesis of Intermediate 35

Intermediate 15 (5.86 g, 17.25 mmol), intermediate 20 (8.16 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 7.07 g (67.6% yield) of intermediate 35.

### Synthesis of Compound 8

Intermediate 35 (3.61 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 8 (0.73 g, 1.03 mmol).

### Synthetic Example 9

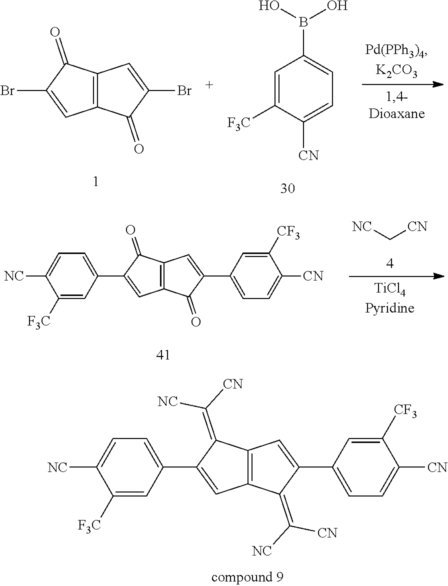 [SMILES: O=c1c(Br)cc2c(=O)c(Br)cc1=2 ; N#Cc1ccc(B(O)O)cc1C(F)(F)F ; N#Cc1ccc(-c2cc3c(=O)c(-c4ccc(C#N)c(C(F)(F)F)c4)cc=3c2=O)cc1C(F)(F)F ; N#CCC#N ; N#CC(C#N)=C1C(c2ccc(C#N)c(C(F)(F)F)c2)=CC2=C1C=C(c1ccc(C#N)c(C(F)(F)F)c1)C2=C(C#N)C#N]

### Synthesis of Intermediate 41

Intermediate 1 (4.97 g, 17.25 mmol) and intermediate 30 (8.16 g, 37.95 mmol) were dissolved in potassium carbonate aqueous solution (2 M, 20 mL) and 1,4-dioxane (60 ml). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 6.52 g (72.6% yield) of intermediate 41.

### Synthesis of Compound 9

Intermediate 41 (2.80 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result was separated with dichloromethane, ethyl acetate, and n-hexane. After that, dichloromethane and petroleum ether were added to the result to form a precipitated liquid, then filtered to obtain compound 9 (0.76 g, 1.34 mmol).

### Synthetic Example 10

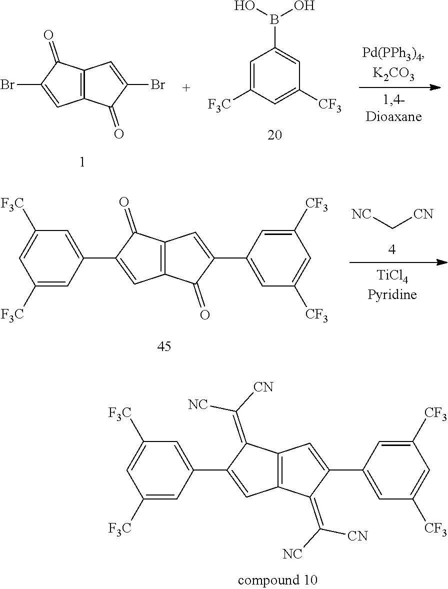 [SMILES: O=c1c(Br)cc2c(=O)c(Br)cc1=2 ; OB(O)c1cc(C(F)(F)F)cc(C(F)(F)F)c1 ; O=c1c(-c2cc(C(F)(F)F)cc(C(F)(F)F)c2)cc2c(=O)c(-c3cc(C(F)(F)F)cc(C(F)(F)F)c3)cc1=2 ; N#CCC#N ; N#CC(C#N)=C1C(c2cc(C(F)(F)F)cc(C(F)(F)F)c2)=CC2=C1C=C(c1cc(C(F)(F)F)cc(C(F)(F)F)c1)C2=C(C#N)C#N]

### Synthesis of Intermediate 45

Intermediate 1 (4.97 g, 17.25 mmol) and intermediate 20 (8.16 g, 37.95 mmol) were dissolved in potassium carbonate aqueous solution (2 M, 20 mL) and 1,4-dioxane (60 mL). After bubbling for 30 minutes, catalyst Pd(PPh₃)₄ (0.30 g) was added to the above mixture in N₂ atmosphere, and the mixture was refluxed for 24 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtain 7.14 g (74.3% yield) of intermediate 45.

### Synthesis of Compound 10

Intermediate 45 (3.31 g, 5.95 mmol) and intermediate 4 (1.17 g, 17.85 mmol) were added to a 250 mL round bottom flask, then the mixture was pour into dichloromethane (250 mL). After cooling down to 0° C., titanium tetrachloride (1.96 mL, 17.85 mol) and pyridine (2.80 mL, 35.70 mol) were added slowly and successively dropwise into the system, then the resulting mixture was stirred at room temperature for 24 h. After the reaction was completed, the obtained crude product was dissolved in dichloromethane and the resulting organic phase was washed with water for three times. After the separation and concentration, the result separated with dichloromethane, ethyl acetate, and n-hexane. After that, dichloromethane and petroleum ether were added to the result to form a precipitated liquid, then filtered to obtain compound 10 (0.79 g, 1.21 mmol).

### Energy Structure of Organic Molecules

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/Semi-empirical/Default Spin/B3LYP” and the basis set “6-31G (d)” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, where S1 and T1 are used directly.

$$HOMO ( eV ) = ( ( HOMO ( G ) \times 27.212 ) - 0.9899 ) / 1 \text{.1206}$$

$$LUMO ( eV ) = ( ( LUMO ( G ) \times 27.212 ) - 2.0041 ) / 1.385$$

Where HOMO(G) and LUMO(G) are the direct calculation results of Gaussian 09W, in units of Hartree. The results are shown in Table 1 below:

TABLE 1

| | Homo Corr. | ^{Δ} Homo Corr. | Lumo Corr. | ^{Δ} Lumo Corr. |
|---|---|---|---|---|
| MatName | [eV] | [eV] | [eV] | [eV] |
| compound 1 | −7.10 | 0.68 | −5.16 | 1.09 |
| compound 2 | −7.65 | 0.01 | −5.44 | 0.97 |
| compound 3 | −6.77 | 0.35 | −4.96 | 1.08 |
| compound 4 | −7.40 | 0.90 | −5.48 | 0.83 |
| compound 5 | −6.83 | 1.12 | −4.88 | 0.86 |
| compound 6 | −6.81 | 0.92 | −5.07 | 0.96 |
| compound 7 | −7.55 | 0.78 | −5.65 | 0.85 |
| compound 8 | −7.44 | 0.94 | −5.52 | 0.86 |
| compound 9 | −7.09 | 0.96 | −5.34 | 1.05 |
| compound 10 | −6.95 | 1.18 | −5.21 | 1.10 |

### Preparation and Characterization of OLED Devices

### Materials Used for Various Layer of OLED Devices

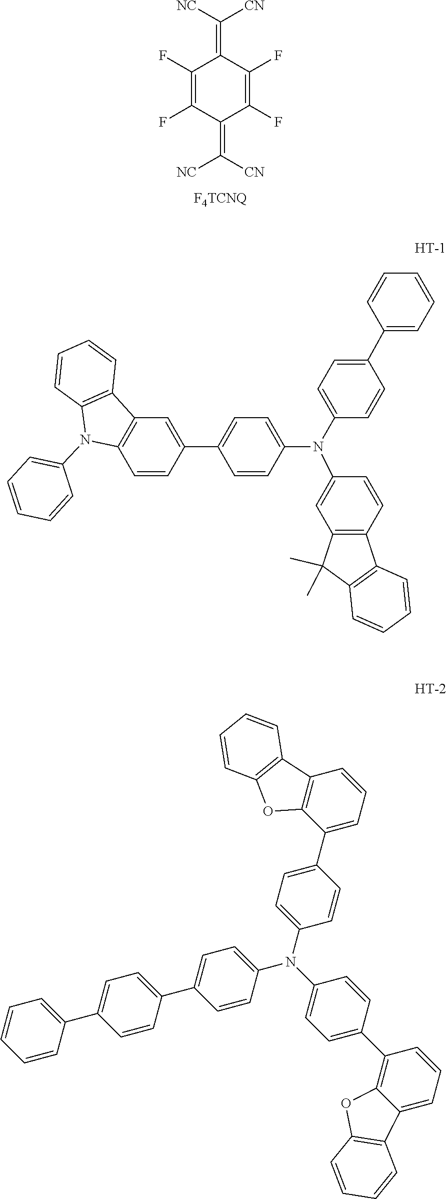 [SMILES: N#CC(C#N)=c1c(F)c(F)c(=C(C#N)C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

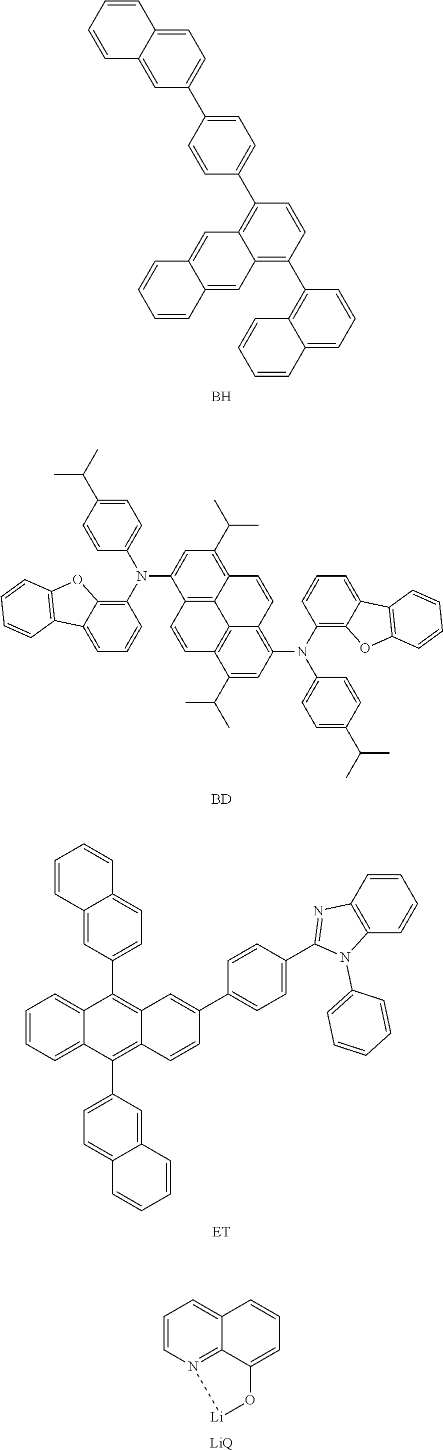

The OLEDs were fabricated based on the device structure of ITO/HI (10 nm)/HT-1 (50 nm)/HT-2 (10 nm)/BH:BD (25 nm)/ET:LiQ (30 nm)/LiQ(1 nm)/Al (100 nm), and the specific preparation procedures are as follows.
a. Cleaning of the conductive glass substrate: prior to first-time use, the substrates are washed with various solvents (such as chloroform, ketone, or isopropyl alcohol), and then treated with UV and ozone.
b. The resultant ITO substrate was mounted on a vacuum deposition apparatus in high vacuum (1×10⁻⁶ mbar), the weight ratio of the compound HT-1 and the compound was controlled to be 98:2 to form a hole-injection layer having a thickness of 10 nm, then the compound HT-1 and the compound HT-2 were vacuum-deposited sequentially on the hole-injection layer to form two hole-transport layers having a thickness of 50 nm and 10 nm, respectively. Then the weight ratio of the BH and the BD was controlled to be 97:3 to form a light-emitting layer having a thickness of 25 nm. Subsequently, the compound ET and LiQ were placed in two different evaporation units, and co-deposited on the light-emitting layer at a weight ratio of 50:50 to form an electron-transport layer having a thickness of 30 nm. LiQ was then deposited on the electron-transport layer to form an electron-injection layer having a thickness of 1 nm, and Al was deposited on the electron-injection layer to form a cathode having a thickness of 100 nm.
c. Encapsulation: encapsulating the device in a nitrogen-regulated glove box with UV curable resin.

All devices have the same embodiments except for the hole-injection layer, which uses a different compound as the dopant. The current-voltage (J-V) characteristics of each OLED were studied. The current efficiency, lifetime and the external quantum efficiency were summarized in Table 2, where the lifetime value is the relative value of the devices with the comparative example 1.

TABLE 2

| | | | | lifetime |
|---|---|---|---|---|
| | | voltage (V) | efficiency | (LT95) |
| | Material | @10 mA/cm² | (cd/A) | @50 mA/cm² |
| Device | compound 1 | 4.3 | 5.9 | 110% |
| Example 1 | | | | |
| Device | compound 2 | 4.1 | 6.0 | 131% |
| Example 2 | | | | |
| Device | compound 4 | 4.0 | 6.3 | 142% |
| Example 3 | | | | |
| Device | compound 7 | 4.0 | 6.5 | 129% |
| Example 4 | | | | |
| Device | compound 8 | 3.9 | 6.6 | 134% |
| Example 5 | | | | |
| Device | compound 9 | 4.0 | 6.2 | 147% |
| Example 6 | | | | |
| Device | compound 10 | 4.3 | 6.3 | 136% |
| Example 7 | | | | |
| Comparative | F4TCNQ | 4.3 | 5.7 | 100% |
| Example 1 | | | | |

As shown in Table 2, the organic compound as described herein is use as a material or the hole-injection layer of the organic electronic device, which leads to a decrease in its operating voltage, and a significant increase in both its efficiency and lifetime relative to comparative example 1. This indicates that the organic compound as described can reduce the injection barrier and better achieve the carrier transport balance, thus realizing the improvement of the efficiency and the lifetime.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. An organic compound, comprising a structure of formula (I-1): 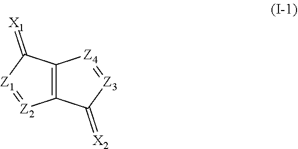
wherein, Z₁-Z₄ are independently selected from CR₁ or N;
X₁ and X₂ are independently selected from CR₂R₃, NR₂, O, S, S(═O)₂, SiR₂R₃, a substituted/unsubstituted aromatic group containing 6 to 60 carbon atoms, or a substituted/unsubstituted heteroaromatic group containing 5 to 60 ring atoms, and at least one of X₁, X₂, Z₁, Z₂, Z₃, or Z₄ comprises a cyano group;
R₁ in multiple occurrences, is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof;
each of R₂ and R₃ at each occurrence is independently selected from the group consisting of a nitro group, a nitroso group, —CF₃, —Cl, —Br, —F, —I, a cyano group, an alkoxy group, a C₁-C₂₀ ketone group, a C₂-C₂₀ alkoxycarbonyl group, a C₇-C₂₀ aryloxycarbonyl group, a carbamoyl group, a haloformyl group, a formyl group, an isocyano group, an isocyanate group, a thiocyanate group, an isothiocyanate group, a hydroxyl group, a substituted/unsubstituted aromatic or heteroaromatic group containing 5 to 60 ring atoms, an aryloxy or heteroaryloxy group containing 5 to 60 ring atoms, and any combination thereof.

2. The organic compound according to claim 1, comprising a structure of one of formulas (II-1)—(II-3): 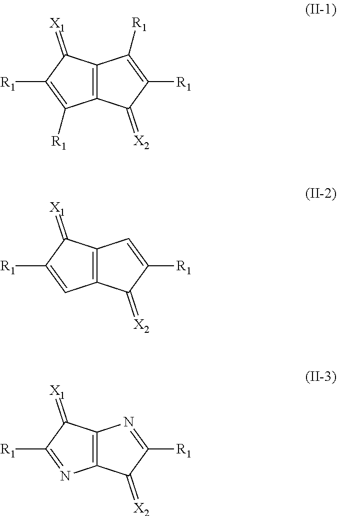
wherein, R₁ in multiple occurrences, is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; X₁ and X₂ are independently selected from CR₂R₃, NR₂, O, S, S(═O)₂, SiR₂R₃, a substituted/unsubstituted aromatic group containing 6 to 60 carbon atoms, or a substituted/unsubstituted heteroaromatic group containing 5 to 60 ring atoms.

3. The organic compound according to claim 1, wherein each of X₁ and X₂ is independently selected from the following groups: 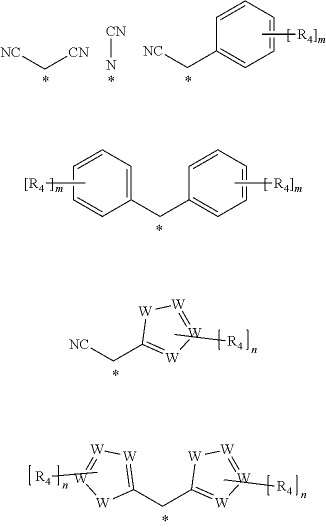
wherein:
R₄ at each occurrence is independently selected from a cyano group, a nitro group, a nitroso group, —CF₃, —Cl, —B, —I, —F, or —OCF₃;
m is an integer from 0 to 5; n is an integer from 0 to 4;
W at each occurrence is independently selected from O, S, or N;
* denotes a linkage site.

4. The organic compound according to claim 2, wherein each of X₁ and X₂ is independently selected from the following groups: 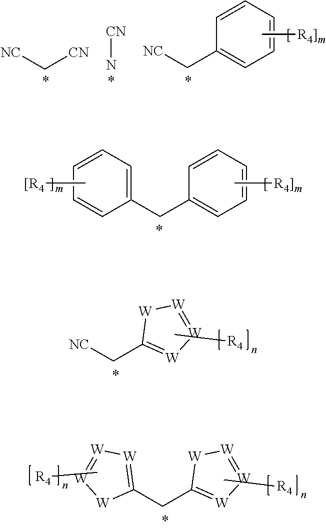
wherein:
R₄ at each occurrence is independently selected from a cyano group, a nitro group, a nitroso group, —CF₃, —Cl, —B, —I, —F, or —OCF₃;
m is an integer from 0 to 5; n is an integer from 0 to 4;
W at each occurrence is independently selected from O, S, or N;
* denotes a linkage site.

5. The organic compound according to claim 1, wherein each of X₁ and X₂ is independently selected from the following groups: 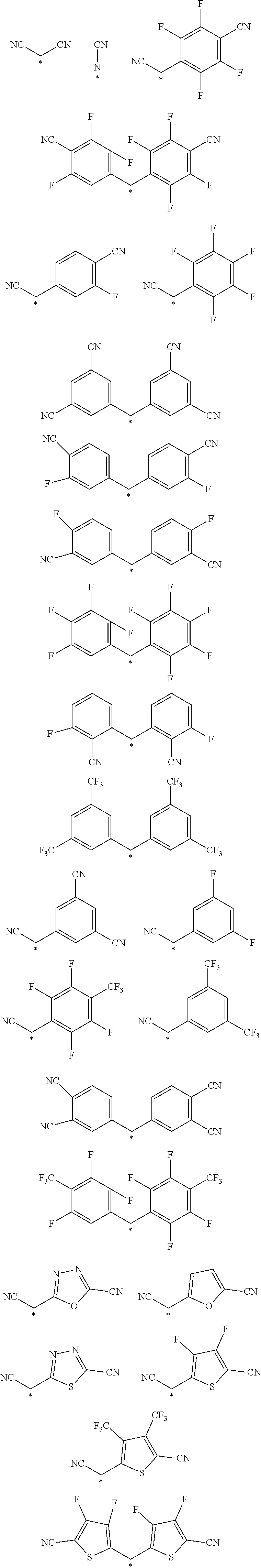
wherein, * denotes a linkage site.

6. The organic compound according to claim 2, wherein each of X₁ and X₂ is independently selected from the following groups: 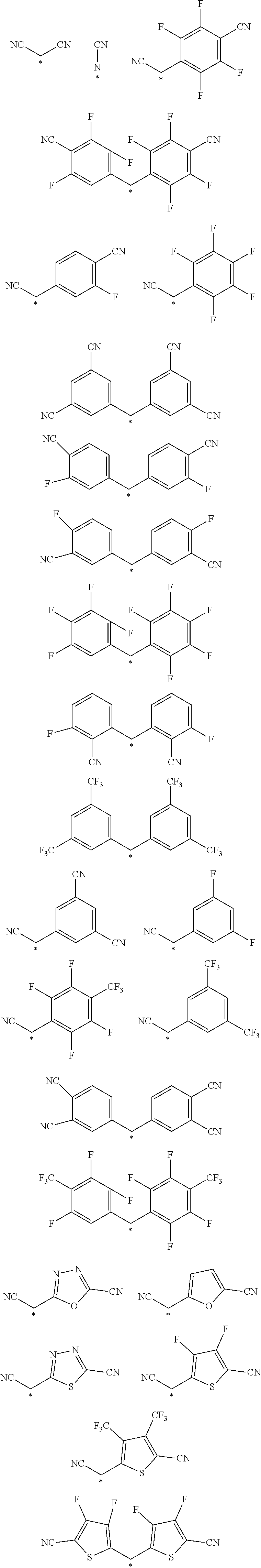
wherein, * denotes a linkage site.

7. The organic compound according to claim 3, wherein each of X₁ and X₂ is independently selected from the following groups: 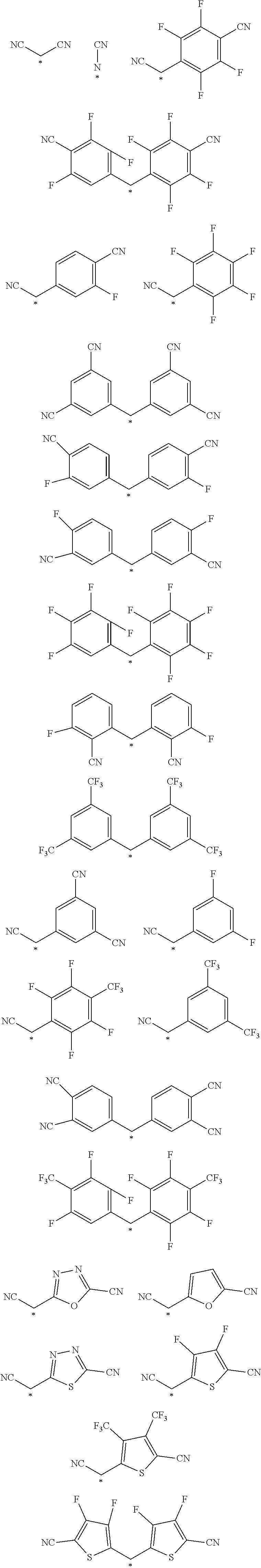
wherein, * denotes a linkage site.

8. The organic compound according to claim 4, wherein each of X₁ and X₂ is independently selected from the following groups: 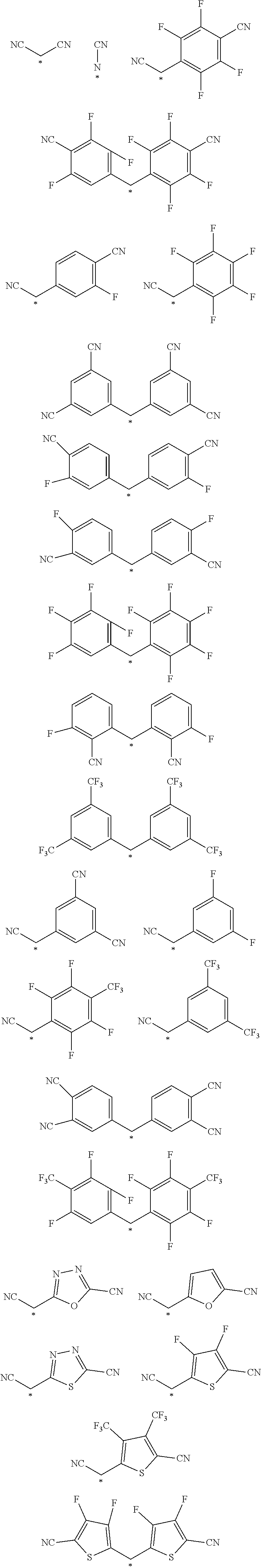
wherein, * denotes a linkage site.

9. The organic compound according to claim 1, wherein each R₁ is independently selected from a cyano group, a nitro group, a nitroso group, —CF₃, —Cl, —B, —I, —F, —OCF₃, or a cyano-, nitro-, nitroso-, CF₃—, Cl—, B—, I—, F—, or OCF₃-substituted aromatic or heteroaromatic group.

10. The organic compound according to claim 2, wherein each RF is independently selected from a cyano group, a nitro group, a nitroso group, —CF₃, —Cl, —B, —I, —F, —OCF₃, or a cyano-, nitro-, nitroso-, CF₃—, Cl—, B—, I—, F—, or OCF₃-substituted aromatic or heteroaromatic group.

11. A mixture, comprising the organic compound according to claim 1, and at least one organic functional material, wherein the at least one organic functional material is selected from a hole-injection material, a hole-transport material, an electron-transport material, an electron-injection material, an electron-blocking material, a hole-blocking material, an emitting material, a host material, or an organic dye.

12. An organic electronic device, comprising a functional layer, wherein the functional layer comprises the organic compound according to claim 1.

13. The organic electronic device according to claim 12, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

14. The organic electronic device according to claim 13, wherein the functional layer is selected from a hole-injection layer or a hole-transport material.

15. An organic electronic device, comprising a functional layer, wherein the functional layer comprises the mixture according to claim 11.

16. The organic electronic device according to claim 15, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

17. The organic electronic device according to claim 16, wherein the functional layer is selected from a hole-injection layer or a hole-transport material.
